SEQUENCE LISTING (9 sequences)

SEQ ID NO: 1 - protein, 11 aa, Artificial Sequence
  Alpha-tubulin peptide
ALEKDYENVGV

SEQ ID NO: 2 - protein, 9 aa, Artificial Sequence
  Synthetic
MXGXGSKCY

SEQ ID NO: 3 - protein, 10 aa, Artificial Sequence
  Synthetic
KGSGCSGSGK

SEQ ID NO: 4 - protein, 7 aa, Artificial Sequence
  Synthetic
ILKDGAC

SEQ ID NO: 5 - protein, 30 aa, Artificial Sequence
  Synthetic
SADSAKDSADSKSADSAKDSADSKADSADK

SEQ ID NO: 6 - protein, 4 aa, Artificial Sequence
  Synthetic
ACYC

SEQ ID NO: 7 - protein, 10 aa, Artificial Sequence
  Synthetic
KGSGCSGSGK

SEQ ID NO: 8 - protein, 10 aa, Artificial Sequence
  Synthetic
KGSGCSGSGK

SEQ ID NO: 9 - protein, 10 aa, Artificial Sequence
  Synthetic
ILKDGACPLI

(19) United States
(12) Patent Application Publication
(10) Pub. No.: US 2015/0087526 A1
(43) Pub. Date: Mar. 26, 2015

(54) PEPTIDE IDENTIFICATION AND SEQUENCING BY SINGLE-MOLECULE DETECTION OF PEPTIDES UNDERGOING DEGRADATION

(71) Applicant: The Regents of the University of colorado, a body corporate, Denver, CO (US)

(72) Inventor: Jay R. Hesselberth, Denver, CO (US)

(21) Appl. No.: 14/374,335

(22) PCT Filed: Jan. 24, 2013
(86) PCT No.: PCT/US13/23002

§ 371 Date: Jul. 24, 2014

Related U.S. Application Data
(60) Provisional application No. 61/589,985, filed on Jan. 24, 2012.

(51) Int. Cl. G01N 33/68; G06F 19/22; G01N 33/58

(52) U.S. Cl. CPC G01N 33/6818; G01N 33/6824; G01N 33/582; G06F 19/22
USPC 506/2; 436/89; 435/23; 435/24; 702/20

(57) ABSTRACT

The present disclosure provides peptide amino acid sequencing and identification methods and kits for performing such methods. For example, single-molecule detection of fluorophore-labeled peptides is disclosed using multiple rounds of standard Edman degradation or using digestion by chemicals or enzymes. Different fluorophores covalently attached to each of a specific type of amino acid side chain of a peptide provide for the derivation of the peptide's encoded amino acid sequence following image alignments of multiple Edman cycles or following digestion by chemicals or enzymes. The amino acid sequence of a peptide and/or the identity of the peptide can be determined by bioinformatic analysis based on the encoded amino acid sequence. The present disclosure further provides peptide derivatization and immobilization strategies to enable the sequencing and identification of a single peptide or a plurality of peptides.

### CROSS-REFERENCE TO RELATED APPLICATION

This application is a 35 U.S.C. §371 national phase application of PCT/US2013/023002 (WO2013/112745), filed on Jan. 24, 2013, entitled “Peptide Identification and Sequencing by Single-Molecule Detection of Peptides Undergoing Degradation”, which application claims priority under 35 U.S.C. §119(e) to U.S. Provisional Application Ser. No. 61/589,985, which was filed on Jan. 24, 2012, disclosures of which are incorporated herein in their entirety.

Incorporated by reference herein in its entirety is the Sequence Listing entitled “Sequence_Listing_ST25.txt”, created Oct. 27, 2014, size of 3 kilobytes.

### FIELD OF THE INVENTION

The present disclosure relates generally to the field of peptide identification and sequencing methods and more particularly to methods comprising differential labeling of amino acids in one or more peptides followed by attachment to a surface, imaging by single molecule detection, cleavage and post-cleavage imaging to identify and sequence one or more peptides. The disclosure further relates to materials for identifying and sequencing peptides.

### BACKGROUND

The following description provides a summary of information relevant to the present disclosure and is not an admission that any of the information provided or publications referenced herein is prior art to the present disclosure.

Unlike the recent massive acceleration realized in DNA sequencing, polypeptide sequencing is a comparatively slow process. Whereas approximately 1 billion 50 base-pair fragments of DNA per day can be sequenced on a single instrument, a single mass spectrometer (MS) is only capable of approximately 100 thousand unique polypeptide sequences. Even with improvements in upstream sample preparation and liquid chromatography, a fundamental speed limit of MS analysis is approaching quickly, such that further increases in the speed of MS polypeptide sequencing will likely be incremental.

At the same time, the development of MS-based proteomics for peptide identification ignited interest in the use of proteins as biomarkers of disease states. Protein and other biomarkers are the foundation of “early detection” strategies geared to identify molecular signatures of disease states prior to their onset. Hartwell L, et al. (2006) Cancer biomarkers: a systems approach. Nat Biotechnol 24: 905-908. A few protein biomarkers are used for cancer and other diagnoses; for example, levels of prostate specific antigen rise during disease progression and are used clinically. Catalona W J, et al. (1991) Measurement of prostate-specific antigen in serum as a screening test for prostate cancer. N Engl J Med 324: 1156-1161. A number of methods have been applied for the identification of new biomarkers, including antibody-based enrichment (Anderson N L, et al. (2004) Mass spectrometric quantitation of peptides and proteins using Stable Isotope Standards and Capture by Anti-Peptide Antibodies (SISCAPA). J Proteome Res 3: 235-244), serum fractionation (Whiteaker J R, et al. (2007) Head-to-head comparison of serum fractionation techniques. J Proteome Res 6: 828-836) and selected ion monitoring (Makawita S, and Diamandis E P (2010) The bottleneck in the cancer biomarker pipeline and protein quantification through mass spectrometry-based approaches: current strategies for candidate verification. Clin Chem 56: 212-222). In addition to new experimental approaches, significant effort is currently focused on optimizing sample collection and the analysis pipeline (Rifai N, et al. (2006) Protein biomarker discovery and validation: the long and uncertain path to clinical utility. Nat Biotechnol 24: 971-983). However, despite some success with a few protein biomarkers, discovery of new biomarkers is limited, due in large part to an inability to efficiently sift through complex peptide mixtures. As a result, the number of new FDA-approved biomarkers has declined over the last decade, and the current rate of biomarker validation is approximately 1 per year (Rifai N, et al. (2006) Nat Biotechnol 24: 971-983).

One approach to advancing biomarker identification is to improve the ability to quantitatively analyze complex protein mixtures. Although mass spectrometers are capable of sequencing full peptides and identifying post-translational modifications, their primary use is in peptide identification. In this mode, an observed mass spectrum is compared against a library of hypothetical mass spectra derived from a protein database. Yates J R, 3rd, et al. (1995) Method to correlate tandem mass spectra of modified peptides to amino acid sequences in the protein database. Anal Chem 67: 1426-1436. However, because of the sequential nature of peptide identification using MS, this process is duty cycle-limited.

Accordingly, what is needed in the art is massively parallel observation of individual peptide sequencing reactions. The principles underlying such a system are analogous to DNA sequencing-by-synthesis, in which DNA sequences are successively built up over multiple cycles of nucleotide incorporation and imaging. For example, on commercial instruments such as the ILLUMINA HISEQ 2000, this process enables the sequencing of 1 billion 50 base-pair fragments in 2 days. Bentley D R, et al. (2008) Accurate whole human genome sequencing using reversible terminator chemistry. Nature 456: 53-59. By spatially separating the sequencing process and observing sequencing reactions independently, extremely large numbers of molecules can be analyzed simultaneously.

### Proteins

Proteins, polypeptides and/or peptides are biochemical compounds comprising a linear polymer chain of amino acid residues that are typically folded into a globular or fibrous form, facilitating a biological function. The linear polymer chain of amino acids comprises an amino acid sequence (i.e., primary structure), wherein the amino acids are bonded together by peptide bonds between the carboxyl and amino groups of adjacent amino acid residues. A peptide bond generally has two resonance forms that contribute some double-bond character and inhibit rotation around its axis, so that the alpha carbons of each amino acid in a polymer chain are roughly coplanar. The other two dihedral angles in the peptide bond determine the local shape assumed by the protein backbone. The end of the protein with a free carboxyl group is known as the C-terminus or carboxy terminus, whereas the end with a free amino group is known as the N-terminus or amino terminus.

The sequence of amino acids in a protein is defined by the sequence of a gene, which is encoded in the genetic code. In general, the genetic code specifies twenty (20) standard amino acids; however, in certain organisms the genetic code can include selenocysteine—and in certain archaea—pyrrolysine. Shortly after, or even during, biological synthesis the residues in a protein are often chemically modified by post-translational modification(s), which alters the physical and chemical properties, folding, stability, activity, and ultimately, the function of the proteins. These post-translational modifications may include, but are not limited to, γ-carboxylation, glycosylation, and/or phosphorylation. Sometimes proteins have non-peptide groups attached, which can be called prosthetic groups or cofactors. One feature of proteins and/or polypeptides comprises an ability to exist in many different conformations. These conformations may be described as: i) secondary structure (e.g., conformations occurring along the dimension of the primary structure including but not limited to beta pleated sheets, alpha helixes, and/or turns); ii) tertiary structure (e.g., conformations comprising folding and/or looping outside the dimension of the primary structure); and iii) quaternary structure (e.g., conformations resulting from interactions between at least two subunits of a polypeptide). Proteins can also work together to achieve a particular function, and they often associate to form stable protein complexes.

Proteins may be purified using a variety of techniques such as ultracentrifugation, precipitation, electrophoresis, and chromatography; and genetic engineering advances have made possible a number of methods to facilitate purification. Methods commonly used to study protein structure and function include but are not limited to immunohistochemistry, site-directed mutagenesis, nuclear magnetic resonance and/or mass spectrometry. Distributed computing can examine complex interactions that govern protein folding, wherein compatible statistical analysis techniques can calculate a protein's probable tertiary structure from its amino acid sequence (primary structure).

Most proteins comprise linear polymers built from series of up to twenty (20) different L-α-amino acids. All proteinogenic amino acids possess common structural features, including an α-carbon to which an amino group, a carboxyl group, and a variable side chain are bonded. Only proline differs from this basic structure as it contains an unusual ring to the N-end amine group, which forces the CO—NH amide moiety into a fixed conformation. The side chains of the standard amino acids have a great variety of chemical structures and properties, wherein it is the combined effect of all of the amino acid side chains in a protein that ultimately determines its three-dimensional structure and its chemical reactivity. Once linked in a protein chain (e.g., protein, polypeptide and/or peptide) an individual amino acid is called a residue, and the linked series of carbon, nitrogen, and oxygen atoms are known as the main chain or protein backbone.

The total complement of proteins present at a time in a cell or cell type is known as its proteome, and the study of such large-scale data sets defines the field of proteomics, named by analogy to the related field of genomics. Useful experimental techniques in proteomics include, but are not limited to: i) two dimensional electrophoresis, which allows the separation of a large number of proteins; ii) mass spectrometry, which allows rapid high-throughput identification of proteins and sequencing of peptides; iii) protein microarrays, which allow the detection of the relative levels of a large number of proteins present in a cell; and iv) two-hybrid screening, which allows the systematic exploration of protein-protein interactions.

A large amount of genomic and proteomic data is available for a variety of organisms, including the human genome (e.g., nucleic acid and/or protein databases). These databases are configured to efficiently identify homologous proteins in distantly related organisms by performing a sequence alignment comparison in response to a sequence query. More sophisticated sequence profiling tools can perform more specific sequence manipulations such as restriction enzyme maps, open reading frame analyses for nucleotide sequences, and secondary structure prediction. As is compatible with some embodiments of the present invention, bioinformatic applications are useful to assemble, annotate, calculate and analyze genomic and proteomic data.

### Edman Protein Degradation

Peptide sequencing using the Edman degradation has been a workhorse of protein biochemistry since its development by Pehr Edman in the 1950's. Edman P (1970) Sequence determination. Mol Biol Biochem Biophys 8: 211-255; Niall H D (1973) Automated Edman degradation: the protein sequenator. Methods Enzymol 27: 942-1010. The chemistry is simple and robust, allowing up to 60 cycles of chemistry to be performed, yielding a peptide sequence, one residue per cycle. See, FIG. 2. Edman sequencing proceeds from the N-terminus of a peptide, which is first derivatized with phenylisothiocyanate (PITC) under moderately basic (pH 8.0) conditions. See, FIG. 2; Reaction 1. The peptide-PITC adduct is then treated with strong acid (e.g. 25% trifluoroacetic acid (TFA), pH 1.5) and heat (50° C.), causing the N-terminal residue to undergo a cyclization and release of a thiazolinone amino acid derivative. See, FIG. 2; Reaction 2. It is believed that this release of the thiazolinone amino acid results in a new amino terminus on the adjacent residue, which is available for derivatization in a subsequent Edman reaction cycle. The thiazolinone amino acid further rearranges into a more stable phenylthiohydantoin (PTH) amino acid derivative and can be isolated by extraction into organic solvent. Finally, the PTH-amino acid derivatives are chromatographically analyzed against standards to identify the residue. The chemistry has been extensively optimized and affords high cleavage efficiencies at each cycle. Niall H D (1973) Methods Enzymol 27: 942-1010. Variants of Edman chemistry have been developed that achieve residue cleavage under mild conditions.

### Amino Acid Sequencing Using Peptide Labels

Previous reports have described various methods of sequencing peptides by attaching labels to the N-terminal amino acid of a peptide and cleaving the peptide, but reports regarding labeling of specific interior amino acids of the peptide and sequencing by cleavage have not been found. Consequently, the reported methods of peptide sequencing by cleavage only provide information regarding the N-terminal amino acid of a peptide, and not to the linked amino acids (e.g., via peptide bonds) that comprise the interior amino acids of an existing peptide. Further, the conventionally known Edman degradation-based methods for sequencing peptides directly detect and identify the released cyclized terminal amino acid.

Methods for sequencing a polypeptide and/or structurally characterizing a polypeptide using labeled N-terminal amino acid specific complexing agents have been reported. Cargile et al., “Concurrent Identification of Multitudes of Polypeptides,” Patent Cooperation Treaty Publication Number WO/2010/065322. These methods relate to using arrays for identifying specific polypeptides of interest from a sample comprising multiple polypeptides where a fluorescent complexing agent (e.g., an antibody) directly labels the N-terminal amino acid of a polypeptide. Consequently, when the N-terminal amino acid is released during Edman degradation, the cyclized amino acid is isolated and directly identified. The residual peptide now has a different N-terminal amino acid that must then be labeled for a successive round of Edman degradation. Direct differential amino acid fluorophore labeling is not performed nor is there any partial sequence identification comparison analysis (e.g., encoded peptides).

Methods have been reported for improving single molecule protein analysis. For example, surface bound peptides may be directly sequenced using a modified Edman degradation wherein each successive amino acid residue is detected by binding to a labeled antibody that is specific for the Edman cyclization product of a terminal amino acid (i.e., a phenylthiocarbamoyl amino acid derivative). Mitra et al., “Single Molecule Protein Screening,” Patent Cooperation Treaty Publication Number WO/2010/065531. Such detection is described as using Total Internal Reflection Fluorescence (TIRF) imaging to produce a “digital profile” that enables protein identification. Direct differential amino acid fluorophore labeling is not performed nor is there any partial sequence identification comparison analysis (e.g., encoded peptides).

Other methods have been described that use alkoxythiocarbonylimidazole derivatization of the N-terminal amino acid residue to perform a modified Edman degradation reaction. These reagents form an alkoxy thiourea derivative that is cleaved with acid to remove the N-terminal amino acid as a stable thiazolinone, which does not rearrange to a thiohydantoin. The thiohydantoin is derivatized with a fluorophore so that the released amino acid residue may be detected. Bailey, J., “N-Terminal Protein Sequencing Reagents and Methods Which Form Amino Acid Detectable by a Variety of Techniques” U.S. Pat. No. 5,807,748 (herein incorporated by reference). Direct differential amino acid fluorophore labeling is not performed nor is there any partial sequence identification comparison analysis (e.g., encoded peptides).

Problems involving post-Edman cycle interference from the presence of residual fluorescence labels, has been addressed by the addition of ammonium salts (e.g., ammonium acetate). Nokihara et al., “Method for Amino Acid Sequence Analysis” U.S. Pat. No. 5,234,836 (herein incorporated by reference). These studies showed that the addition of ammonium salts to standard N-terminal fluorescent labeling of a peptide did not interfere with the Edman reaction or subsequent identification of the released residue.

Protein sequencing methods have been reported that first modify the protein by reducing cysteine disulphide bridges, digesting the protein into peptides and then labeling the lysine residues with mass tags. The peptides are then sequenced using mass spectrometry. Hamon et al., “Method for Characterizing Polypeptides,” European Patent EP1397686B1. Direct differential amino acid fluorophore labeling is not performed nor is there any partial sequence identification comparison analysis (e.g., encoded peptides).

Mass spectrometry has also been used with chemically modified proteins to generate an amino acid sequence where fluorescent labeling is not used. Such chemicals that modify the amino acid side chains include: N-hydroxysuccinimide, N-(p-(2-benzoxazolyl)phenyl) maleimide, and/or 1-ethyl-3-(3-dimethylaminopropyl)-carbodiimide hydrochloride (EDC). Fluorophores are not described in the reference. Schneider et al., “Methods for Sequencing Proteins” U.S. Pat. No. 6,716,636 (herein incorporated by reference). Direct differential amino acid fluorophore labeling is not performed nor is there any partial sequence identification comparison analysis.

Thus, in view of the foregoing, a need persists in the art for faster, more accurate protein identification and sequencing methods.

### SUMMARY OF THE INVENTION

The present disclosure provides peptide identification and sequencing methods which may comprise differential labeling of amino acids of a peptide; attachment of a peptide to a surface; imaging of a peptide by single molecule detection; cleavage of a peptide by Edman degradation, enzymatic digestion or chemical digestion; post-cleavage imaging of a peptide by single-molecule detection; and determination of peptide identity or sequence based on changes in the peptide image pre-cleavage and post-cleavage. Included are materials and kits for preforming peptide identification and sequencing methods.

### BRIEF DESCRIPTION OF THE FIGURES

FIG. 1 presents exemplary data showing the simulation of recovery of unique peptides (dashed) and proteins (solid) from the Uniprot collection of human proteins. Different levels of recovery are observed by adding residues to the collection that are labeled. Left Panel: Labeling of lysine (K). Middle Panel: Labeling of lysine and cysteine (KC). Right Panel: Labeling of lysine, cysteine, tyrosine, and tryptophan (KCYW).

FIG. 2 presents a representative schematic of a conventional Edman protein degradation cycle.

FIG. 3A presents an overview of fluorophore derivatization, immobilization and single molecule detection. A peptide I-L-K-D-G-A-C-P-L-I (SEQ ID No: 9) is derivatized with two distinct fluorophores, immobilized for single molecule detection and detected.

FIG. 3B presents an overview of single molecule Edman peptide sequencing. During Edman sequencing, the peptide loses fluorophore-derivatized amino acid residues at specific cycles, allowing assignment of those residues in an encoded sequence that can be used for subsequent database matching.

FIG. 3C presents an overview of single molecule peptide identification by digestion. During peptide identification by digestion, the peptide loses fluorophore-derivatized amino acid residues after digestion resulting in an optical transition from one combination of fluorophores before digestion to a second, possibly different combination of fluorophores after digestion. These “optical transitions” can be can be used for subsequent database matching.

FIG. 4 presents a representative counting and imaging device compatible with the methods of the current invention. The device performs Total Internal Reflection Fluorescence (TIRF) and collects an image of the differentially labeled peptides and counts the individual fluoresence probes per peptide, thereby providing spatial information regarding the specific amino acid sequence.

FIG. 5 presents a representative embodiment of how the TIRF technique works when detecting and counting the fluorescent probes in various embodiments of the present invention.

FIG. 6A presents C-terminal labeling of a model peptide (Angiotensin II) with a biotin-PEG moiety using oxazolone chemistry.

FIG. 6B presents validation of C-terminal biotin-PEG attachment using MALDI mass spectrometry. The mass signature at 1074 m/v corresponds to formylated Angiotensin II, a side product of the oxazalone activation chemistry.

FIG. 7A presents C-terminal labeling of a model peptide (Angiotensin II) with a Click chemistry-compatible DBCO moiety using oxazalone chemistry.

FIG. 7B presents validation of C-terminal DBCO attachment using MALDI mass spectrometry. The mass signature at 1074 m/z corresponds to formylated Angiotensin II, a side product of the oxazalone activation chemistry.

FIG. 8A presents image collected of alpha-tubulin peptides lacking C-terminal biotin moieties (110 features counted).

FIG. 8B presents image collected of alpha-tubulin peptides with C-terminal biotin moieties (3,050 features counted); this represents a 30-fold increase in the number of molecules immobilized upon biotin derivatization, illustrating the currently achievable signal-to-noise attributed to specific derivatization.

FIG. 8C presents specific immobilization of peptides on a solid surface for single molecule detection. Alpha-tubulin peptide with sequence NH2-A-L-E-K-D-Y-E-N-V-G-V (SEQ ID No: 1) was derivatized at its lysine residue with NHS-ALEXA 555, followed by either no treatment, or derivatization at its C-terminus with biotin using oxazalone chemistry (e.g. FIG. 6A). Immobilization of the peptides via streptavidin linkage to flow cells enables their visualization by TIRF microscopy.

FIG. 9A presents analysis of sequential digests of a peptide (described in FIG. 11 and legend). Quantitative comparison of images in FIGS. 9B and 9C shows that >90% of the molecules are cleaved by trypsin, losing their ALEXA 555 fluorophores. Minimal background is observed for dye-labeled peptides that lack C-terminal biotin moieties (slanted lines; 14 molecules counted in a single field)

FIG. 9B presents imaged field of biotinylated peptides with ALEXA 555 fluorophores (5,156 features counted).

FIG. 9C presents imaged field of peptides from FIG. 9B pre-treated with trypsin, liberating the ALEXA 555 molecules (485 features counted).

FIG. 10A presents analysis of sequential digests of a peptide (described in FIG. 11 and legend). Quantitative comparison of images in FIGS. 10B and 10C shows that most of the molecules retain ALEXA 647 upon trypsin digestion. Minimal background is observed for dye-labeled peptides that lack C-terminal biotin moieties (slanted lines; 19 molecules counted in a single field).

FIG. 10B presents imaged field of biotinylated peptides with ALEXA 647 fluorophores (265 features counted).

FIG. 10C presents imaged field of peptides from FIG. 10B pre-treated with trypsin, liberating the ALEXA 555 molecules (417 features counted).

FIG. 11 presents example of sequential digestion of peptides showing loss of signal following trypsin digestion. A synthetic peptide with sequence NH2-acetyl-M-K(N3)-G-K(N3)-G-S-K-C-Y (SEQ ID No: 2) was first derivatized with a maleimide-ALEXA 647 fluorophore (black spot). The peptide was subsequently derivatized by oxazalone chemistry at its C-terminus with biotin (e.g. FIG. 6A), and finally derivatized by copper-mediated Click chemistry with alkyne-ALEXA 555 (dotted spots). Prior to immobilization and image analysis, an aliquot of the peptide was treated with trypsin, which cleaves at the intervening lysine residue. Thus, a cleavage reaction yields a discernible change in the peptides imaged. Data corresponding to this experiment is shown in FIG. 9A-C and FIG. 10A-C.

FIG. 12 presents example of Edman degradation using Barrett's modification. A peptide with sequence NH2-K(A647)-G-S-G-C-S-G-S-G-K(biotin)-amide (SEQ ID No: 3) was treated with 5 cycles (20 min each) of N-terminal derivatization with 0.1 M phenylisothiocyanate in triethylammonium acetate pH 8.5, followed by analysis of an aliquot by isolation with streptavidin magnetic beads and fluorescence measurement. Over 5 cycles, nearly 50% of the peptides with native N-termini undergo loss of ALEXA 647 signal, indicating removal of the N-terminal residue (NH2, dashed line). An identical peptide with an N-acetyl group is protected from Edman degradation and does not lose fluorescence through 5 cycles (Ac, solid line).

FIG. 13 presents a synthetic peptide with N-terminal acetylation, ALEXA 647-derivatized lysine at residue 1, and C-terminal biotin moiety added to the final lysine residue. This peptide, as well as an identical peptide lacking the N-terminal acetylation, was used for the Edman degradation experiment in FIG. 12

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art(s) to which this invention belongs. Although any methods, devices, and materials similar or equivalent to those described herein can be used in the practice or testing of the invention, the preferred methods, devices and materials are now described.

All publications, published patent documents, and patent applications cited in this disclosure are indicative of the level of skill in the art(s) to which the disclosure pertains. All publications, published patent documents, and patent applications cited herein are hereby incorporated by reference to the same extent as though each individual publication, published patent document, or patent application was specifically and individually indicated as being incorporated by reference.

As used in this disclosure, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged.

The term “encoded state” as used herein, refers to any unambiguous identification of a particular amino acid as a result of losing a fluorescent signal from that particular amino acid during an Edman degradation cycle.

The term “encoded peptide” as used herein, refers to any peptide having at least one unambiguous identification of a particular amino acid.

The terms “differentially labeled amino acid residues”, “differential labeling”, and “differentially labeled” as used herein, refer to a plurality of amino acid residues wherein at least two of the residues are attached to a different label (e.g. a fluorescent label). Differential labeling refers generally to the use of a combination of types of detectable moieties, wherein each type of detectable moiety is specific to an amino acid type. By way of non-limiting example, amino acids of one type (e.g. lysines) are labeled with one detectable moiety (e.g. NHS fluorophore) and amino acids of a different type (e.g. cysteines) are labeled with a different detectable moiety (e.g. a maleimide fluorophore).

The term “component” as used herein, refers to any compound and/or molecule, organic and/or inorganic, that participates in a multi-step chemical reaction (e.g., an Edman degradation reaction).

The term “counting device” as used herein, refers to any device capable of detecting, distinguishing and/or enumerating labels. For example, a counting device may image a differentially labeled peptide such that each different label may be uniquely detected, distinguished and enumerated (i.e., counted). Such a counting device may be part of an imaging device or separate.

The term “terminal amino acid” as used herein, refers to any amino acid residue that comprises a single peptide bond. For example, a C-terminal amino acid has a peptide bond comprising only the amino end, whereas an N-terminal amino acid has a peptide bond comprising only the carboxyl end.

The term “residual peptide” as used herein, refers to a peptide that has been subjected to at least one cycle of Edman degradation. Consequently, the residual peptide is at least one amino acid residue shorter in length than the initial peptide.

The term “solid substrate” as used herein, refers to any surface to which a protein or peptide to be sequenced can be attached either covalently or non-covalently (e.g. immobilized). Various materials may be used including but not limited to polyvinylidene fluoride, glass fiber filters, silica beads, polyethylene, carboxyl modified polyethylene, and/or porous polytetrafluoroethylene.

The terms “arrays” and “microarrays” as used herein are used somewhat interchangeably differing only in general size, and refer to any solid substrate capable of immobilizing a peptide. Each array typically contains many “spots” (typically 100-1,000,000+) wherein each “spot” is at a known or random, arbitrary location and contains a single immobilized peptide. Therefore, each microarray can immobilize many different peptides having many different sequences.

The terms “image”, “imaging”, and “change in the image” as used herein refer to the collection of electromagnetic data emitted by an object (e.g. a protein or peptide). The electromagnetic emission of an object may be a fluorescence emission, radioactive emission, or other electromagnetic emission. The collection of electromagnetic data in the form of an image by an imaging process can be conducted by any method known in the biological, chemical and physical sciences. Known imaging processes include but are not limited to total internal reflection fluorescence (TIRF) microscopy, fluorescence resonance energy transfer microscopy (FRET), multiphoton detection, polarization detection, plasmonic effects detection, atomic force spectroscopy, fluorescence lifetime, light scattering and Raman scattering. A change in an image refers to any change in the electromagnetic data collected for an object from one time point to another time point and an absence of a change in an image refers to any aspect of the electromagnetic data collected from an object that is constant from one time point to another time point.

As used herein, the term “polypeptide” refers generally to a molecule that comprises one or more amino acid monomers covalently linked together. “Polypeptide” includes proteins as well as short polypeptides that are approximately 100 amino acids or less in length. In one embodiment, the polypeptide is 10 amino acids or greater in length. Polypeptides may be artificially synthesized, isolated from nature or modified for compatibility with the methods herein described (e.g., the polypeptide may be digested with trypsin to reduce its size, or other enzymes may be added to remove polysaccharides, neutralizing by mild acid or neuraminidase to remove sialic acid, reacted with alkaline phosphatase to remove phosphate, or with sulfatases or by chemical means to remove sulfate or oxidize thiols).

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “an isolated amino acid”, as used herein, refers to any amino acid molecule that has been removed from its natural state (e.g., removed from a cell and is, in a preferred embodiment, free of other peptides, proteins and/or polypeptides).

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

As used herein, the term “aromatic side chain amino acids” refers a group amino acids, less than all of the amino acids, having a common side chain chemical or structural relationship comprising an aromatic ring substituent (e.g. a benzyl ring). For example, the side chains of amino acid residues histidine, phenylalanine, tryptophan, and tyrosine are structurally related as having an aromatic ring substituent.

As used herein, the term “acidic side chain amino acids” refers a group amino acids, less than all of the amino acids, having a common side chain chemical or structural relationship comprising an acidic group substituent (e.g. a hydrogen donating group). For example, the side chains of amino acid residues aspartic acid and glutamic acid residues are chemically related as having an acidic group substituent.

As used herein, the term “basic side chain amino acids” refers to a group of amino acids, less than all of the amino acids, having a common side chain chemical or structural relationship comprising a basic group substituent (e.g. a hydrogen acceptor group). For example, the side chains of amino acid residues asparagine, glutamine, lysine, arginine, and histidine are chemically related as having a basic group substituent.

As used herein, the term “hydrophobic side chain amino acids” refers to a group of amino acids, less than all of the amino acids, having a common side chain chemical or structural relationship comprising an aliphatic group substituent. For example, the side chains of amino acid residues glycine, alanine, valine, leucine, isoleucine, methionine and proline are chemically related as having an aliphatic group substituent.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derivative” as used herein, refers to any chemical modification of a nucleic acid or an amino acid. Illustrative of such modifications would be replacement of hydrogen by an alkyl, acyl, or amino group.

The terms “label”, “detectable label”, and “detectable moiety” are used herein, to refer to any composition detectable by spectroscopic, photochemical, biochemical, immunochemical, electrical, optical or chemical means. Such labels include biotin for staining with labeled streptavidin conjugate, magnetic beads (e.g., DYNABEADS), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The terms “selective label” and “selectively labels” refer to attachment of a detectable moiety to a particular type of amino acid side chain. Generally, selective labels only label one type of amino acid side chain (e.g. lysine). Yet, in some circumstances selective labels may label multiple types of amino acid side chains that are closely structurally related. By way of non-limiting example, the same selective label may label both aspartate and glutamate side chains which are both negatively charged.

The term “type of amino acid”, as used herein, refers to a particular structure of amino acid wherein all amino acids of a particular type have the same side chain structure.

The term “fluorescence”, as used herein, refers to any process of emitting electromagnetic radiation (light) from an object, chemical and/or compound. Consequently, fluorescence is considered to be a form of luminescence. In most cases, emitted light has a longer wavelength, and therefore lower energy, than the absorbed radiation. However, when the absorbed electromagnetic radiation is intense, it is possible for one electron to absorb two photons; this two-photon absorption can lead to emission of radiation having a shorter wavelength than the absorbed radiation. The emitted radiation may also be of the same wavelength as the absorbed radiation, termed ‘resonance fluorescence”.

The term “fluorescence emission signature”, as used herein, refers to a combination of fluorescence emitted, as well as an absence of fluorescence emission, by a differentially fluorescently labeled protein or peptide. The fluorescence emission signature can refer to the fluorescence emitted or not emitted, by a whole protein, a peptide, a portion of a peptide, or a single residue of a protein or peptide at any position. A fluorescence emission signature can be experimentally determined or can be inferred based on the amino acid sequence of a protein or peptide and differential labeling strategies. By way of non-limiting example, the fluorescence emission signature can be a prediction based on the number of lysines in a protein, if lysines were labeled with a particular fluorophore, and further based on the number of cysteines in a protein, if cystines were labeled with a particular fluorophore distinct from the fluorophore used to label lysines.

In contrast to the protein sequencing and identification methods discussed in the Background of the Invention section above, the present disclosure describes use of peptide labels which covalently, differentially label types of amino acid side chains. In contrast to the vast majority of peptide sequencing methods, the present disclosure describes peptide sequencing and identification methods that do not comprise utilizing affinity reagents (e.g. antibodies). As discussed further below, the use of labels that covalently bond amino acid side chains is superior to labeling of amino acids with affinity reagents because affinity reagents are more susceptible to low binding affinities or off-target binding. Further, covalent labels provide a more robust label attachment that is far less likely to be undesirably disassociated.

The present disclosure provides peptide identification and sequencing methods. Different labels may be attached to specific amino acid side chain types such that differential labeling by type of amino acid is provided. The differentially labeled peptides may then be derivatized for attachment to a surface to facilitate the sequencing of peptides derived from a protein mixture, which mixture is optionally obtained from a biological sample. The peptide's encoded amino acid sequence may be derived by imaging, optionally by single molecule detection, before cleavage; imaging following subsequent rounds of Edman cycles or following digestion by chemical or enzymatic means; and image alignment to detect changes in the image such as loss of a fluorescent label after a given Edman degradation cycle or a given digestion.

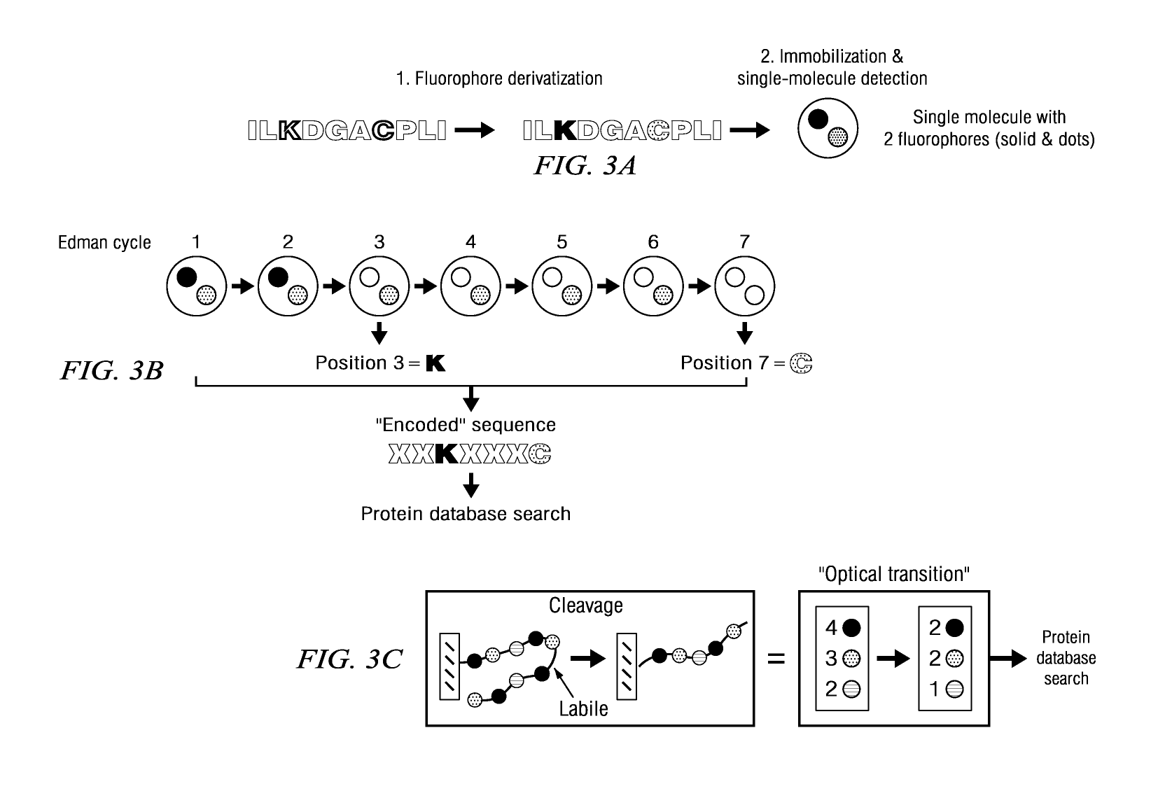

A critical innovation of the present disclosure is that peptides of a given sequence, after differential labeling, have a finite numbers of labels (e.g. fluorophores). Cycles of site-specific digestion of these peptides generate a new set of labels (e.g. fluorophores), which are imaged to count the labels (e.g. fluorophores) remaining after digestion. The changes in the set of labels (e.g. fluorophores) on a peptide, before and after digestion, result in “optical transitions” (FIG. 3C) that can be matched to a protein sequence database with high accuracy. This method draws on the variety of digestion chemistries, and enzymatic strategies, available for peptides. For example, cyanogen bromide cleaves C-terminal of methionine residues; 2-Nitro-5-thiocyanobenzoate (NTCB) cleaves N-terminally of cysteine residues; asparagine-glycine dipeptides can be cleaved using hydroxlamine; and BNPS-skatole cleaves C-terminal of tryptophan residues. The variety of digestion options enables the exploration of combinations of digestion types (i.e. sequential digestion) that yield the most informative set of optical transitions.

Simulations of this process on 20,331 human proteins in the UniProt database ((2010) The Universal Protein Resource (UniProt) in 2010. Nucleic Acids Res 38: D142-148.), show that after NTCB cleavage on peptide molecules derived from cyanogen bromide cleavage and multiply labeled on lysine and cysteine residues, ˜3% of the optical transitions are uniquely identifiable, and 1,412 proteins have at least 1 uniquely identifiable peptide. Two approaches can further improve identification: 1) increasing the number of labeled residues (e.g. including cysteine, tryptophan, tyrosine and glutamate/aspartate residues), or 2) increasing the number of sequential cleavages, producing richer optical transitions. For example, if both lysine and tyrosine residues (Joshi N S, et al. (2004) A three-component Mannich-type reaction for selective tyrosine bioconjugation. J Am Chem Soc 126: 15942-15943; Tilley S D and Francis M B (2006) Tyrosine-selective protein alkylation using pi-allylpalladium complexes. J Am Chem Soc 128: 1080-1081) are labeled, 12% of the optical transitions derived from BNPS cleavage of CNBr-peptides are uniquely identifiable, and 65% of proteins contain a uniquely identifiable peptide; adding an additional NTCB cleavage results in 14% of the encoded sequences are uniquely identifiable (71% of proteins). Other labeling strategies may also be used such as chemoselective strategies for labeling tryptophan (Antos J M, et al. (2009) Chemoselective tryptophan labeling with rhodium carbenoids at mild pH. J Am Chem Soc 131: 6301-6308) can be added, enabling highly sensitive peptide and protein detection. Alternatively, ambiguous labeling of glutamate and aspartate could be employed (using EDC activation and fluorophore-hydrazides), which significantly improves detection limits.

It is believed, by some skilled artisans in the field, that a massive acceleration in the rate of peptide sequencing would transform proteomics research. To accomplish this, some embodiments of the present invention contemplate a highly parallel peptide sequencing platform based on single molecule detection of individual, labeled (e.g., fluorescently labeled) peptides undergoing Edman degradation or sequential cleavage. This platform leverages existing, commercially available technology, yielding a conceptually simple and widely applicable method for multiplexed peptide sequencing. In addition to proteomic applications (e.g. cancer research) the sequencing platform is extensible to many applications, thereby allowing comprehensive and quantitative peptide identification on a scale that has not previously been achievable.

The massively parallel peptide sequencing technology disclosed herein can sequence huge numbers of peptides derived from a complex protein mixture (e.g. whole proteome sequencing). In one embodiment disclosed herein, peptide sequences are generated in a reduced representation in which the positions of a specific subset of amino acid side chains are known (e.g. encoded sequences). These “encoded” sequences can be used for protein database searches to identify their matching peptide sequences. Some embodiments disclosed herein leverage several existing and proven methods to produce a new technology suitable for large-scale protein and peptide sequencing.

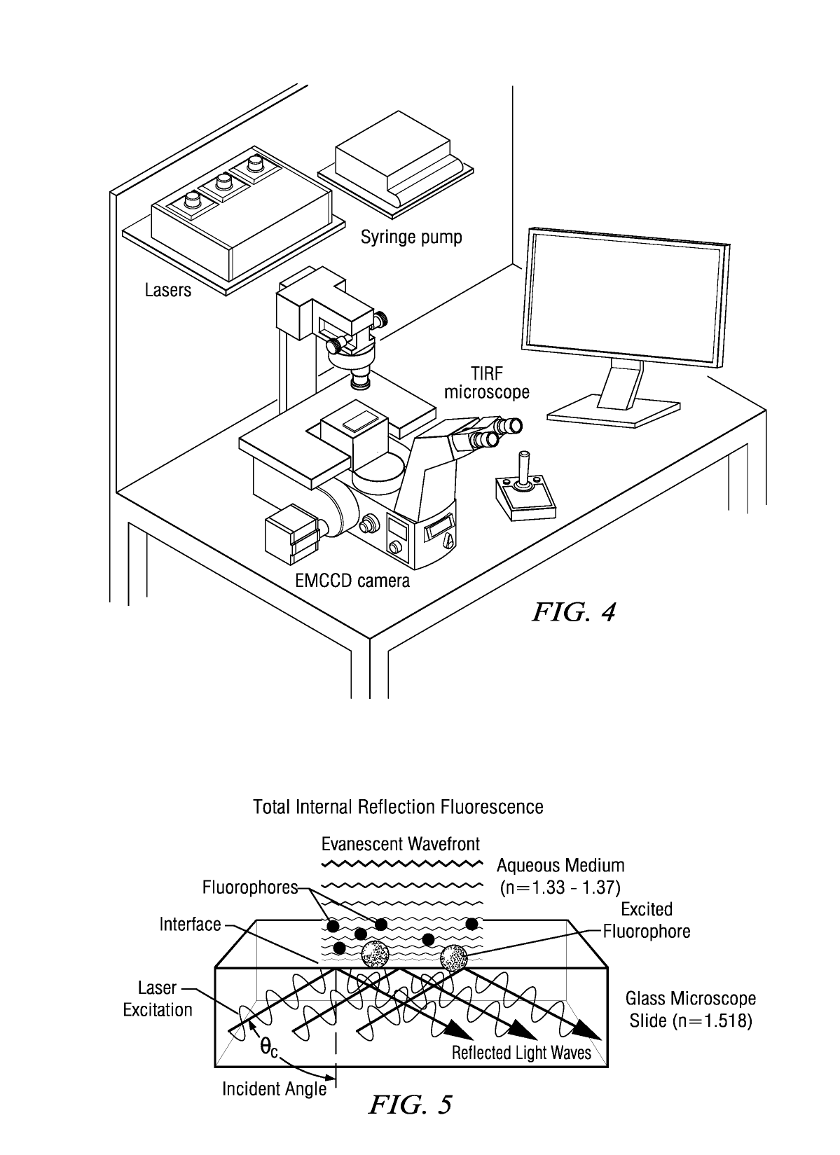

The present disclosure describes methods for identification and sequencing polypeptides wherein, in one aspect, each of at least two types of amino acid side chain are selectively attached to one different label per type of amino acid labeled. In one embodiment, peptides with specific fluorophore-derivatized amino acids are immobilized on the surface of a cover slip in preparation for single-molecule detection by total internal reflection fluorescence (TIRF) microscopy. See, FIG. 4. Specific amino acids within a peptide are derivatized with fluorophores based on existing chemistries (e.g. NHS-fluorophores react with the primary amine of lysine and maleimide-fluorophores react with the thiol of cysteine). In one embodiment, these peptides are subjected to multiple rounds of Edman degradation, which result in the loss of at least one (1) labeled amino acid residue from the N-terminus of each peptide per cycle. The loss of a fluorescently labeled amino acid residue results in a loss of fluorescence on the peptide for that specific amino acid and/or side chain, allowing an unambiguous assignment of the residue and/or side chain based on which fluorophore was lost (i.e. if a fluorophore derivatized to lysine is lost, assign lysine to this position). Similarly, the absence of a loss of a fluorescently labeled amino acid residue from the peptide following a cycle of Edman degradation indicates that a fluorophore derivatized amino acid was not lost due to the cycle of Edman degradation providing at least some information regarding the character of the amino acid cleaved by the Edman degradation.

Edman sequencing of proteins yields sequence information—i.e. the linear arrangement of amino acids in the peptide. The encoded sequences derived from image analysis in our system are used in an alignment step to identify probable peptide sequence matches. A typical 30 position sequence will contain 5-10 sites where the residue is known unambiguously, and other positions will be “placeholder” positions, i.e. the identity of the residue at this position is not known definitively, but cannot be one of the residues that was initially modified. Thus, the identities of the known residues as well as their relative positions are informative and can be used during sequence alignment. Alignment of this encoded sequence to a sequence database is analogous to peptide or DNA sequence matching to a sequence database, except the encoded sequences contain extensive missing information. A standard protein sequence database (e.g. Uniprot protein sequences) is used for this purpose, assuming that the peptides in the sample derive from this database.

Encoded sequences can be used in existing dynamic programming sequence alignment algorithms (e.g. Smith-Waterman) to identify probable matches in a protein sequence database. These algorithms will treat “placeholder” positions as neutral with regard to the scoring matrix, such that typical scores from an alignment traceback will be lower than similar traditional sequence alignment approaches. Statistical approaches permit a robust alignment in the face of false-positive “insertions” and “deletions” created by inefficient derivatization or Edman cleavage.

In the sequential cleavage approach, counts of fluorophores are obtained after each cleavage step. Using a protein sequence database, the counts are used to match peptides, given the numbers of fluorophores (i.e. composition of labeled amino acids) and the chemistry that matches particular cleavage steps. For example, after a cyanogen bromide digest, peptides containing methionine will be cleaved, resulting in the loss of a fragment with some number of fluorophores. The difference in the number of fluorophores before and after this cleavage is thus used for matching.

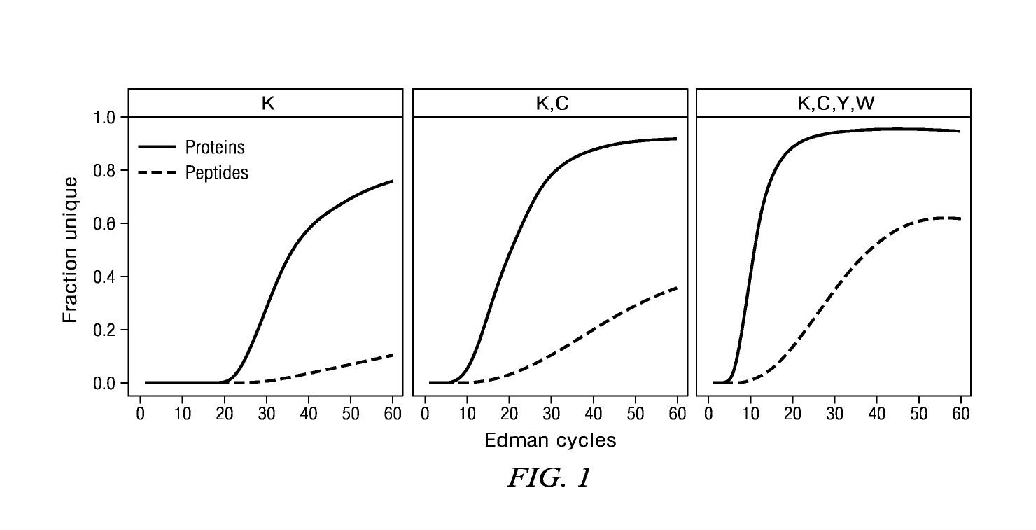

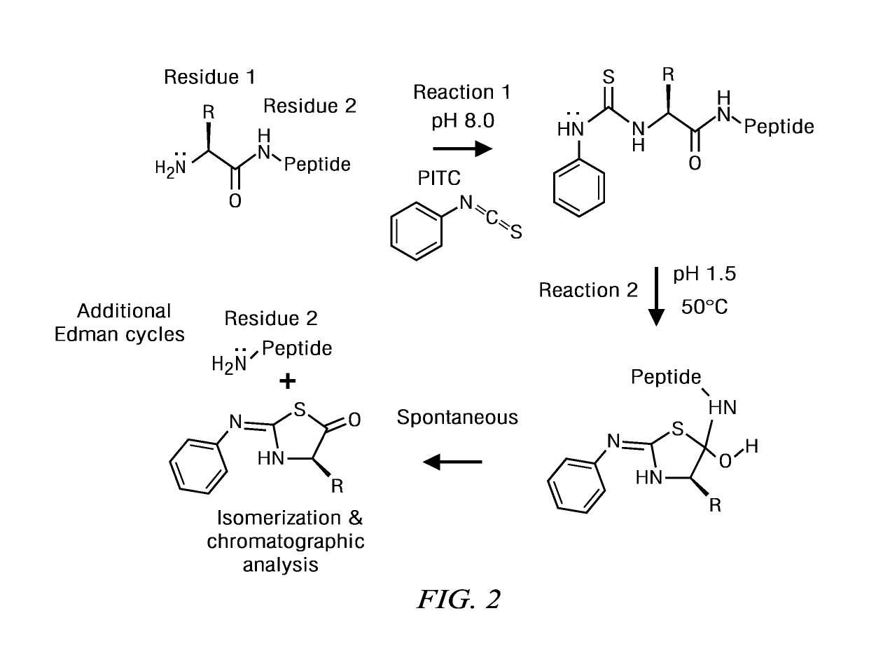

In one embodiment, multiple Edman cycles (e.g. thirty to sixty (30-60)) comprising repetitive derivatization and degradation of a peptide are capable of identifying an encoded amino acid sequence. For example, a partial intermediate sequence (e.g., an encoded amino acid sequence) may be represented as, XXXKXXXCXXXKKXXXC, where C and K are the positions of fluorescently labeled residues and X represents non-labeled amino acid positions. Although the present disclosure is not to be limited by any particular mechanism, it is believed that, given enough cycles, this intermediate sequence may be uniquely identifiable from a comparison to a database of existing protein sequences. For example, by using derivatized lysine (K) and cysteine (C) residues, 50 cycles of Edman degradation allows the detection of ˜50% of peptides in the human proteome (and ˜75% percent of proteins). If one additionally labels tryptophan (W), almost 50% of the proteome can be detected at thirty (30) cycles (FIG. 1).

The present method has significant advantages over other, more conventional, amino acid sequencing methods including, but not limited to: i) optical detection of single molecules that increases the speed with which it allows one to sequence peptides; and ii) a large increase in the number of peptides sequenced when integrated with massively parallel technology. Currently, standard methods for sequencing polypeptides usually rely upon techniques such as liquid chromatography and/or mass spectrometry. Even the most advanced of these conventional sequencing techniques is capable of only sequencing ten-fifty thousand (10-50,000) peptides per day. It is believed that the massively parallel advantages disclosed herein is capable of sequencing 10-100 million peptides/day, thereby representing a ten thousand fold (10,000-fold) increase in peptide sequencing speed.

### Direct Differential Side Chain Labeling

In some embodiments, the present invention contemplates peptide sequencing systems based on single molecule detection (SMD) of fluorophore-derivatized polypeptides undergoing cycles of Edman degradation. In this system, peptides to be sequenced are first derivatized with amino acid-reactive fluorophores (i.e. fluorophores are covalently bonded to the side chains of certain amino acids comprising the peptides). Following immobilization (e.g. on a solid substrate comprising glass and/or silicon) a plurality of peptides undergo repetitive Edman degradation cycles in combination with SMD. This system and method generates peptide sequences in an encoded state. In general, an encoded state is defined as an unambiguous identification of a particular amino acid residue as a result of losing a fluorescent signal associated with that particular amino acid during an Edman degradation cycle. Such an amino acid identification can be made by comparing at least two images of the labeled peptide; a first image taken before the Edman cycle and a second image taken after the Edman cycle. Preliminary simulation studies have shown that following approximately thirty (30) Edman degradation cycles on lysine-derivatized peptides within the Uniprot human protein database, the method would identify at least 20% of the encoded 30-residue peptide sequences. See, FIG. 1, left panel. It was further found that this analysis also finds approximately 8,000 proteins having at least one (1) uniquely identifiable peptide.

Direct differential amino acid side chain labeling for determining peptide amino acid sequences offers several advantages over mass spectrometry (MS)-based peptide sequencing platforms. Most notably, direct differential amino acid side chain labeling is contemplated as capable of sequencing between approximately ten million-five hundred million (10-500 million) peptides per day, preferably between approximately fifty million-three hundred million (50-300 million) peptides per day, and more preferably between approximately seventy-five million-one-hundred and fifty million (75-150 million) peptides per day. Consequently, the present method would be expected to yield between approximately 100-5,000-fold the number of peptides sequenced using a conventional mass spectrophotometric-based technology. This surprising advance in the high-throughput capacity of peptide sequencing is expected to transform capabilities for identifying protein and peptide diagnostic biomarkers (e.g. early detection of cancer) and/or a vast improvement in the efficiencies of proteomic studies. In addition, direct differential amino acid side chain labeling is conceptually simple; may employ off-the-shelf components and reagents, may rely on total internal reflection fluorescence (TIRF) microscopy for single molecule-sensitivity, and is supported by the reliability of Edman chemistry and conventional sequence comparison algorithms.

In one embodiment, the present invention contemplates a highly multiplexed system for sequencing individual peptides. For example, peptides are first derivatized with commercially available, amino acid-reactive fluorophores: e.g. lysine side chains may be labeled via their primary amines with N-hydroxysuccinimide (NHS) chemistry, and cysteine side chains may be labeled via their thiols using maleimide chemistry. For example, the peptide NH₂-ILKDGAC-COOH (SEQ ID No: 4) would be labeled with one dye on its lysine residue, and a second, spectrally distinct dye on its cysteine. See, FIG. 3A. Once labeled, the peptides are immobilized on a glass cover slip for single molecule detection. Following immobilization, the first two steps of Edman degradation (e.g. PITC-derivatization and cleavage) are performed on the peptides to sequentially remove residues from their N-termini. At the end of each cycle, cleaved PTH-amino acid moieties are washed away, and an image of each residual labeled peptide, as a single molecule, is collected. By tracing the cleavage pattern observed for each single molecule, an “encoded” peptide sequence is generated, one residue per cycle.

Although the present disclosure is not to be limited by any particular mechanism, it is believed that cycles in which a fluorophore is lost from a single molecule allow assignment of that residue in the peptide sequence, and cycles that do not remove a fluorophore assign positions that were not labeled. See, FIG. 3B. For example, after 7 cycles of Edman chemistry, the sequence XXKXXXC would be generated. These “encoded” sequences can provide sufficient information to allow their identification by matching the sequence to a peptide sequence database.

Approximately 20,331 human proteins have been accumulated in the UniProt database. (2010) The Universal Protein Resource (UniProt) in 2010. Nucleic Acids Res 38: D142-148. The data presented herein illustrate the results of simulations of differential amino acid side chain labeling simulations performed on the UniProt database (FIG. 1). The results indicated that after 30 cycles of Edman sequencing on single molecules with fluorophore-derivatized lysine residues, 5% of the 30-residue “encoded” sequences are uniquely identifiable, and >2,000 proteins have at least 1 uniquely identifiable peptide. Peptide identification can be further improved by: 1) increasing the number of labeled residues (e.g. including lysine, cysteine, tyrosine and tryptophan residues); and 2) increasing the number of Edman cycles, thereby producing longer encoded sequences. See, FIG. 1, right panel. For example, if both lysine and cysteine are labeled, 18% of the 30-residue encoded peptide sequences are uniquely identifiable, and 40% of proteins contain a uniquely identifiable peptide; for 60-residue peptides, 60% of the encoded sequences are uniquely identifiable (83% of proteins). See, FIG. 1, middle panel. Alternatively, chemoselective strategies for labeling tyrosine (Joshi N S, et al. (2004) A three-component Mannich-type reaction for selective tyrosine bioconjugation. J Am Chem Soc 126: 15942-15943; Tilley S D and Francis M B (2006) Tyrosine-selective protein alkylation using pi-allylpalladium complexes. J Am Chem Soc 128: 1080-1081) and tryptophan (Antos J M, et al. (2009) Chemoselective tryptophan labeling with rhodium carbenoids at mild pH. J Am Chem Soc 131: 6301-6308) may be employed, thereby improving the sensitivity of peptide and protein detection at 30 Edman cycles.

### Detecting Fluorophore-Labeled Single Molecules

In one embodiment, the present invention contemplates detecting single-molecule fluorophore-labeled synthetic peptides following exposure to multiple rounds of Edman sequencing chemistry. In one embodiment, the method comprises stabilizing the fluorophores in various Edman chemical schemes. In one embodiment, the method comprises counting small numbers of fluorophores present in single molecules.

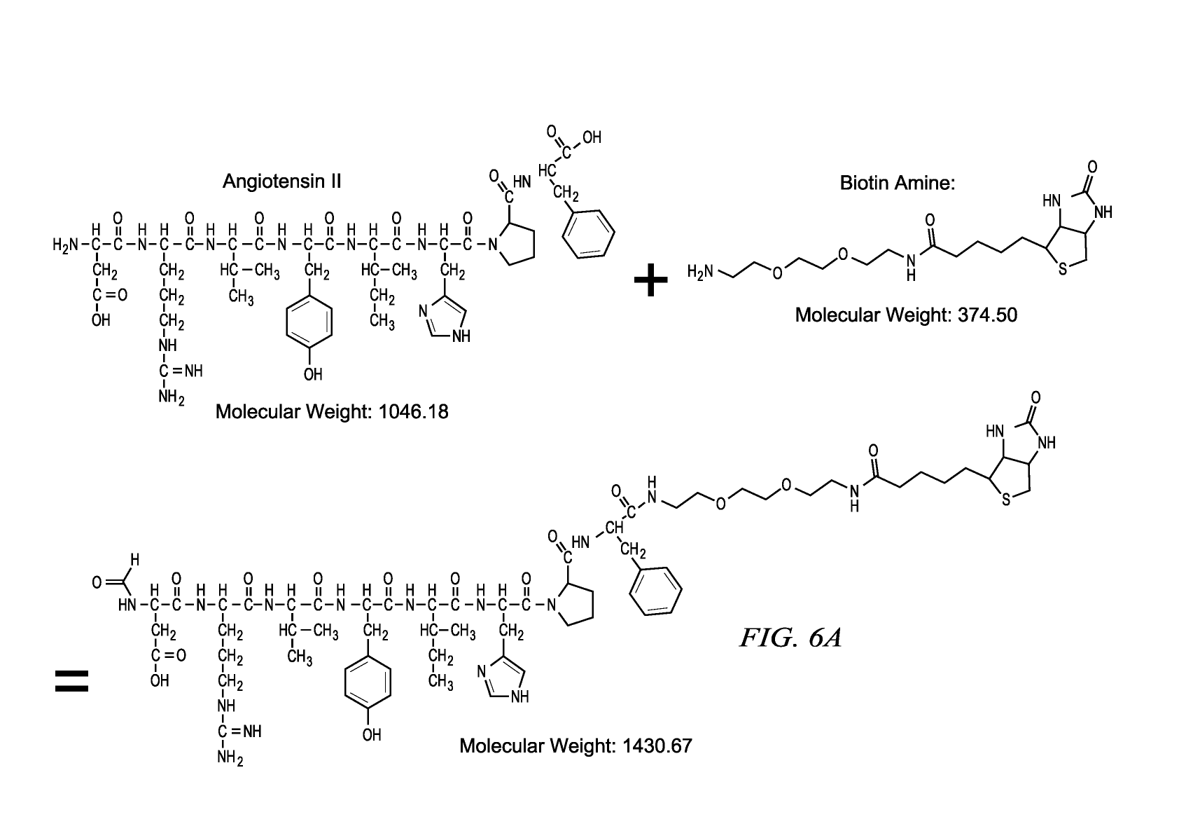

In one embodiment of SMD experiments, single molecules may be monitored using Total Internal Reflection Fluorescence (TIRF) microscopy. In general, TIRF microscopy comprises an excitation laser that illuminates a substrate at a critical angle, thereby exciting fluorophores within 100-300 nm of the substrate surface. Although the present disclosure is not to be limited by any particular mechanism, it is believed that fluorophore photon emission is then captured using sensitive electron-multiplied charge-coupled device (EMCCD) cameras, resulting the detection of 1,000-10,000 single fluorophores in an optical field. See, FIG. 5. With commercially available TIRF microscopes and off-the-shelf fluidic systems, SMD experiments are becoming routine, enabling many types of biochemical measurements at the single-molecule level (e.g. processivity of RNA polymerases (Galburt E A, Grill S W, Bustamante C (2009) Single molecule transcription elongation. Methods 48: 323-332), tRNA selection by the ribosome (Blanchard S C, et al. (2004) tRNA dynamics on the ribosome during translation. Proc Natl Acad Sci USA 101: 12893-12898) and mRNA splicing (Abelson J, et al. (2010) Conformational dynamics of single pre-mRNA molecules during in vitro splicing. Nat Struct Mol Biol 17: 504-512)). For example, a useful TIRF microscopy setup has been previously reported for the single-molecule detection of proteins comprising a Nikon TIRF microscope (NIKON USA, Inc.), with a fluidic system enabling flow of reagents over single molecules immobilized on cover slips (BIOPTECHS, Inc.). Tessler L A, et al. (2009) Protein quantification in complex mixtures by solid phase single-molecule counting. Anal Chem 81: 7141-7148.

### Fluorophore Stability Through Multiple Cycles of Edman Chemistry

In some embodiments, SMD may be performed using peptides derivatized with amino acid-reactive fluorescent dyes. Following derivatization, the peptides will be immobilized on a solid surface (e.g. silicon, glass and/or quartz) and subjected to multiple rounds of Edman degradation. Edman degradation may be performed with alternating treatments of phenylisothiocyanate in a mildly basic solution (0.1 M TEA, pH 8.0), followed by strong acid (25% trifluoroacetic acid, ˜pH 1.5). Each of these treatments (e.g., cycles) may be at least one (1) minute in length at ambient temperatures. Preferred fluorescent dyes exhibit robust photostability after exposure to Edman degradation, and are not reactive with the PITC derivatization reagent.

Some commercially available dyes may have sufficient stability to withstand multiple Edman sequencing cycles. For example, the ALEXA FLUOR series, several dyes that lack exocyclic sulfonic acid groups are stable at pH 1 (INVITROGEN, INC., personal communication). Panchuk-Voloshina N, Haugland R P, Bishop-Stewart J, Bhalgat M K, Millard P J, et al. (1999) ALEXA dyes, a series of new fluorescent dyes that yield exceptionally bright, photostable conjugates. J Histochem Cytochem 47: 1179-1188. In addition, the HYLYTE dye series is stable at low pH (ANASPEC, INC., personal communication), providing another alternative for peptide labeling. In addition, none of these dyes contain primary amines, precluding their reaction with PITC during Edman cycles. These commercially available fluorophores may be evaluated by subjecting them to multiple rounds of Edman degradation and monitoring their photostability (e.g. fluorescence intensity and photobleaching rates). One method may involve labeling primary-amine-coated magnetic beads with NHS-fluorophore derivatives (e.g., ALEXA FLUOR 568, 594 and 633). The NHS-flurophore labeled primary amine magnetic beads can then be treated with either PITC in 0.1 M TEA (pH 8.0), or 25% TFA (pH 1.5) for 5 minutes (i.e., the conditions from a single Edman cycle). After magnetic isolation, the beads are washed with neutralizing buffer and their bulk fluorescence measured to determine their photostabilities. The photostability of fluorophores can also be determined for up to 30 sequential cycles (PITC/pH 8.0 followed by pH 1.5) of Edman degradation. It has been found that several commercially available fluorophores maintain photostability following multiple rounds of Edman exposure. Other approaches to the Edman degradation use mildly basic solutions to afford cleavage, preserving the nature of most fluorophores (Barrett G C, et al. (1985) Edman Stepwise degradation of polypeptides: a new strategy employing mild basic cleavage conditions. Tetrahedron Letters 26: 4375-4378).

To evaluate fluorophore stability at the single molecule level, 30-residue peptide containing five lysine residues can be synthesized, wherein each are separated by six intervening residues (e.g. NH2-SADSAKDSADSKSADSAKDSADSKADSADK-COOH (SEQ ID No: 5)). In addition, a hydrazino-nicotinamide moiety is incorporated at its C-terminus, facilitating chemoselective immobilization on 4-formylbenzamide-coated cover slips (SOLULINK, INC.). Finally, an additional, nearly identical peptide is synthesized that is blocked at its N-terminus (e.g. acetylation or formylation, ANASPEC, INC.), preventing degradation upon exposure to Edman sequencing cycles. These peptides are then labeled using commercially available NHS-derivatives of any variety of fluorophores and purified to homogeneity using reverse phase HPLC.

Initially, the N-terminally blocked peptides, which do not undergo Edman degradation, are immobilized on quartz cover slips via their C-termini, and imaged using SMD. Photostability of 1,000 individual peptide molecules may be monitored throughout multiple Edman cycles. After each cycle, the number of observable single molecules are measured and quantified to determine their fluorescence intensities and/or photobleaching rates. Optimization of Edman chemistry can identify the best trade-off between fluorophore stability and residue cleavage efficiency. Traditional Edman chemistry employs an ˜10 minute PITC derivatization step under mildly basic conditions, followed by a ˜10 minute treatment in strong acid to cause cyclization and cleavage of the N-terminal residue. Therefore, each cycle ranges between approximately 1 and 10 minutes, and photostabilities can be analyzed against Edman exposure times. Based on these measurements, determine stability may be determined during active Edman sequencing. Subsequent to immobilization of the labeled test peptides with native N-termini it may be determined when fluorophores are lost at pre-determined cycles. For example, when the first of the five lysines is lost, the fluorescence intensity of each molecule in the field should be reduced by ˜20%, confirming that the fluorophores exhibit photostability.

### Counting Multiple Fluorophores in a Single Peptide Molecule

In one embodiment, the present invention contemplates peptides labeled at specific residues with unique fluorophores, wherein a single residue may comprise multiple identical fluorophores. In order to reliably determine when a fluorophore is lost in an Edman cycle, the number of fluorophores present in a single molecule are determined in a given cycle, followed by the number of fluorophores present in the subsequent cycles. For example, in a test peptide, five lysine residues are labeled. Before any Edman cycles, five fluorophores are present in the single residue. However, following cycle 6, 1 fluorophore would be lost and 4 would remain. Therefore, one can distinguish between 5 and 4 fluorophores in this molecule by comparing two separate cycles. In order to estimate how many unique residues, and thereby identical fluorophores, would be present in peptides derived from a complex mixture, the human Uniprot database was examined. It was determined that the 30-residue peptide set derived from this database has a median of 5 lysine and 5 cysteine residues. Statistically, therefore, an ideal method could robustly distinguish between 1 and 5 fluorophores on each single molecule.

A number of strategies may be used to count the number of fluorophores on a multiply labeled single molecule. One approach is to integrate the fluorescence intensities from a collection of single molecules in an optical field, fit a Gaussian to the distribution of intensities, and then calculate the probability of a single molecule containing a quantized number of fluorophores using its observed intensity and the Gaussian fit. Mutch S A, Fujimoto B S, Kuyper C L, Kuo J S, Bajjalieh S M, et al. (2007) Deconvolving single-molecule intensity distributions for quantitative microscopy measurements. Biophys J 92: 2926-2943. Alternatively, fluorophores can be counted by sequentially photobleaching a field by incrementally increasing excitation intensity and observing how many fluorophores remain in a collection of single molecules following each photobleaching step. This approach has been used successfully to count subunits in individual protein complexes (Ulbrich M H and Isacoff E Y (2007) Subunit counting in membrane-bound proteins. Nat Methods 4: 319-321) and to measure sub-wavelength distances between dyes (Gordon M P, et al. (2004) Single-molecule high-resolution imaging with photobleaching. Proc Natl Acad Sci USA 101: 6462-6465). At the single molecule level, several dyes exhibit reversible photobleaching, enabling multiple measurements to be made on individual dyes (Baddeley D, et al. (2009) Light-induced dark states of organic fluochromes enable 30 nm resolution imaging in standard media. Biophys J 96: L22-24).

A preferred method is to use fluorescence intensity integration to establish a counting method for identifying multiply labeled single molecules. For example, test peptide variants may be synthesized and purified that contain between 1 and 5 labeled lysine residues. Equimolar mixtures of these peptide variants are immobilized for SMD, and fluorescence intensities are collected for approximately 1,000 molecules. Known methods may then be applied to quantify numbers of fluorophores for each molecule in the collection (Mutch S A, et al. (2007) Biophys J 92: 2926-2943). Peptide mixtures with other known compositions (e.g. 1 and 2 fluorophores, and 4 and 5 fluorophores) may also be immobilized and measured as controls to determine reliability.

### Image Alignment to Track Single Molecule Positions

In one embodiment, the present invention contemplates a method comprising aligning images acquired during 30 Edman cycles to track the positions of single molecules, such that their encoded sequences may be derived. For example, computational approaches can be developed for tracking the positions of single molecules in a collection of images acquired after each of 30 cycles of Edman sequencing. Previously developed methods for tracking the position of molecules through a series of images, by calculating the cross-correlation between a query and reference image have been reported. Bentley D R, et al. (2008) Accurate whole human genome sequencing using reversible terminator chemistry. Nature 456: 53-59. This method allows the positions of illuminated pixels through a stack of images to be determined, and is robust to identify small changes in the X and Y directions from image-to-image.

In one embodiment, the present invention contemplates tracking the positions of approximately 1,000 single molecules in a single frame throughout 30 cycles of Edman sequencing chemistry. Fluorescent images may be collected after every cycle and subsequently analyzed to track the positions of each single molecule. Optimizing the cross-correlation on the N-terminally blocked synthetic peptide may be performed by collecting images after each of 30 cycles of Edman chemistry. For example, the cross-correlation of each image relative to cycle 1 can then be calculated, and the positions of each molecule from each cycle calculated in the X and Y directions. These offsets are used to calculate the path of each molecule through the image stack. Approximately 30 cycles of sequencing may be performed on the test peptide with a native N-terminus, and when the 1,000 molecules are tracked through cycle 30 the fifth lysine residue is lost and molecules become invisible.

A common problem with the cross-correlation approach comprises “phasing”, in which molecules that do not undergo efficient cleavage become “out-of-phase” relative to the majority of molecules. These “out-of-phase” molecules can generate encoded sequences that contain apparent insertions. In one embodiment, the present invention addresses this problem by using a dynamic programming algorithm to perform gap-tolerant local alignments of encoded sequences to a peptide sequence database. Smith T F and Waterman M S (1981) Identification of common molecular subsequences. J Mol Biol 147: 195-197.

### Peptide Derivatization and Immobilization

In one embodiment, the present invention contemplates a method comprising derivatizing unique amino acids and immobilizing specific peptides derived from a protein mixture.

Optimizing derivatization and immobilization strategies for sequencing proteolytic peptides derived from a simple starting protein mixture comprising an equimolar mixture of 48 human proteins with a wide range in molecular weight. (e.g., SIGMA UNIVERSAL PROTEOMICS STANDARD 1 (UPS1), SIGMA CHEMICAL, INC.). For example, a random mix of peptides can be generated from this mixture by digestion with Proteinase K, and labeled with amine-reactive fluorophores to specifically label lysine residues. Finally, the fluorophore-labeled peptides are purified by bulk reverse phase chromatography.

Strategies for immobilizing peptides on cover slips suitable for TIRF microscopy may then be evaluated. Peptides are covalently attached to silica cover slips and subjected to robust attachment chemistries available for specific amino acid side chains. This allows the peptides to be identified through multiple cycles of Edman chemistry and imaging. Initially, the feasibility of immobilizing peptides can be evaluated via their cysteine thiols to maleimide-derivatized cover slips (ERIE BIOSCIENCES). As an alternative to thiol-based immobilization, peptides may be covalently attached via acidic groups (glutamate, aspartate and the peptide C-terminus), using the water-soluble carbodiimide EDC followed by immobilization on hydrazine-coated cover slips at low pH (pH 5.0). These strategies can be evaluated by imaging a single frame containing 1,000 molecules through 30 cycles of Edman chemistry. The positions of single molecules from each image will then be tracked throughout the experiment using cross-correlation relative to the first collected image. The immobilization strategy that minimizes single molecule movement will be selected for further sequencing.

Approximately, 30 cycles of Edman sequencing can then be performed on a single field containing 1,000 single molecules derived from the peptide mixture. After each cycle, the number of fluorophores are counted in each single molecule, and an examination of each molecule in the image stack identifies the cycles in which a fluorophore was lost. Finally, this information is used to build encoded sequences for each single molecule. Based on the composition of the protein mixture, it is estimated that at least 25 30-residue encoded sequences are uniquely identifiable, and therefore, in one embodiment, we should be able to robustly determine the identities of ˜25 molecules from the imaged field.

### Optional Improvements

In one embodiment, the method further comprises scaling detection and imaging to approximately 10⁶-10⁸ residues by raster-scanning a larger field of single resides and storing the images for each field. It is believed that commercial next-generation DNA sequencers are compatible with improved detection and imaging technology. (e.g. ILLUMINA HISEQ 2000 can sequence ˜1 billion individual clusters in 2 days).

In one embodiment, the method further comprises quantitating the data to normalize the peptide counts by simultaneously analyzing known quantities of synthetic peptide standards. An analogous approach has been used to quantify RNA transcript abundances spanning five (5) orders of magnitude in mRNA-seq experiments, which is similar to the dynamic range exhibited by proteomic methods involving affinity reagents (e.g. proximity ligation). Mortazavi et al., “Mapping and quantifying mammalian transcriptomes by RNA-Seq” Nat Methods 5:621-628 (2008). This issue can be experimentally addressed using the SIGMA UPS2 PROTEOMICS DYNAMIC RANGE STANDARD (SIGMA, INC.), wherein peptide concentrations span five (5) orders of magnitude.

In one embodiment, the method further comprises sample multiplexing to quantitate and validate changes in protein abundance across hundreds or thousands of samples. It is believed that multiplexing greatly facilitates biomarker studies. For example, sample multiplexing naturally allows the parallel analysis of multiple samples (e.g. provided in separate microfluidic flow chambers) analogous to strategies employed in next-generation DNA sequencers.

In one embodiment, the method further comprises post-translational peptide modifications thereby allowing protein modification analysis employing selective enrichment and/or derivatization. For example, phosphopeptides may be isolated prior to sequencing 6, and sites of glycosylation will be directly identified by periodate oxidation of sugar moieties 7 and derivatization with fluorophore hydrazides. Villen et al., “The SCX/IMAC enrichment approach for global phosphorylation analysis by mass spectrometry” Nat Protoc 3:1630-1638 (2008); and Zhang et al., “Identification and quantification of N-linked glycoproteins using hydrazide chemistry, stable isotope labeling and mass spectrometry” Nat Biotechnol 21:660-666 (2003).

The present disclosure, in one embodiment, provides a method for sequencing a peptide comprising: (a) labeling the amino acid side chain of one or more amino acid of a first type with a first detectable moiety, wherein said first detectable moiety selectively labels the side chain characterizing said one or more amino acid of a first type; (b) labeling the amino acid side chain of one or more amino acid of a second type with a second detectable moiety, wherein said second detectable moiety selectively labels the side chain characterizing said one or more amino acid of a second type; (c) attaching said peptide to a surface; (d) imaging said peptide; (e) cleaving said peptide; (f) imaging said peptide after the cleavage of step (e); (g) repeating steps (e) to (f) as necessary; (h) comparing the image of step (d) with the image of step (f) and identifying a change or an absence of a change in the image between step (d) and step (f); (i) if further cleavage is performed as in step (g), comparing the image before and after each subsequent cleavage step (e) and identifying a change or an absence of a change in the image; and (j) determining the sequence of the peptide based on at least one change or at least one absence of a change in the image identified in step (h) or (i).

The present disclosure further provides said immediately preceding method for sequencing a peptide, in one embodiment, wherein before step (c) labeling the amino acid side chain of one or more amino acid of a third type with a third detectable moiety, wherein said third detectable moiety selectively labels the side chain characterizing said one or more amino acid of a third type; in a further embodiment, and wherein before step (c) labeling the amino acid side chain of one or more amino acid of a fourth type with a fourth detectable moiety, wherein said fourth detectable moiety selectively labels the side chain characterizing said one or more amino acid of a fourth type; in a further embodiment, and wherein before step (c) labeling the amino acid side chain of one or more amino acid of a fifth type with a fifth detectable moiety, wherein said fifth detectable moiety selectively labels the side chain characterizing said one or more amino acid of a fifth type; and in a further embodiment, and wherein before step (c) labeling the amino acid side chain of one or more amino acid of a sixth type with a sixth detectable moiety, wherein said sixth detectable moiety selectively labels the side chain characterizing said one or more amino acid of a sixth type.

The present disclosure further provides said immediately preceding method for sequencing a peptide, in one embodiment, wherein the side chain characterizing said one or more amino acid of a first type is positively charged; in a further embodiment, wherein said one or more amino acid of a first type is lysine; in a further embodiment, wherein the side chain characterizing said one or more amino acid of a first type is negatively charged; in a further embodiment, wherein the side chain characterizing said one or more amino acid of a first type is aromatic; in a further embodiment, wherein the side chain characterizing said one or more amino acid of a first type is polar; and in a further embodiment, wherein said one or more amino acid of a first type is cysteine.

The present disclosure further provides said immediately preceding method for sequencing a peptide, in one embodiment, wherein the cleavage of step (e) is Edman degradation; in one embodiment, wherein the cleavage of step (e) is a digestion; and in one embodiment, wherein the digestion is chemical digestion or enzymatic digestion.

The present disclosure further provides said immediately preceding method for sequencing a peptide, in one embodiment, wherein the attaching said peptide to a surface of step (c) is attachment of the C-terminus or a side chain of said peptide to the surface; in one embodiment, wherein each of said detectable moieties is selected from the group consisting of a fluorophore, a dye, a quantum dot, a radiolabel, an enzyme and an enzyme substrate; in one embodiment, wherein each of said detectable moieties is a fluorophore; and in one embodiment, wherein after step (i) and before step (j), comparing at least one change or at least one absence of a change in the image identified in step (h) or (i) to a database of fluorescence emission signatures of known protein sequences, further wherein at least one fluorescence emission signature, or part thereof, is the same as the at least one change or the at least one absence of a change in the image of step (j) used for determining the sequence of the peptide.

The present disclosure provides, in one embodiment, a method for sequencing a plurality of peptides comprising: (a) for each peptide of the plurality, labeling the amino acid side chain of one or more amino acid of a first type with a first detectable moiety, wherein said first detectable moiety selectively labels the side chain characterizing said one or more amino acid of a first type; (b) for each peptide of the plurality, labeling the amino acid side chain of one or more amino acid of a second type with a second detectable moiety, wherein said second detectable moiety selectively labels the side chain characterizing said one or more amino acid of a second type; (c) attaching each of said plurality of peptides to a surface such that each peptide is spatially separated enough to allow single-molecule detection; (d) imaging each of said plurality of peptides using single-molecule detection; (e) cleaving each of said plurality of peptides; (f) imaging each of said plurality of peptides using single-molecule detection after the cleavage of step (e); (g) repeating steps (e) to (f) as necessary; (h) comparing the image of step (d) for each of said plurality of peptides with the corresponding image of step (f) and identifying a change or an absence of a change in the image between step (d) and step (f); (i) if further cleavage is performed as in step (g), comparing the image before and corresponding image after each subsequent cleavage step (e) for each of said plurality of peptides and identifying a change or an absence of a change in the image; and (j) determining the sequence of each of said plurality of peptides based on at least one change or at least one absence of a change in the image as identified in step (h) or (i).

The present disclosure further provides said immediately preceding method for sequencing a plurality of peptides, in one embodiment, wherein before step (c) for each peptide of the plurality, labeling the amino acid side chain of one or more amino acid of a third type with a third detectable moiety, wherein said third detectable moiety selectively labels the side chain characterizing said one or more amino acid of a third type; in a further embodiment, and wherein before step (c) for each peptide of the plurality, labeling the amino acid side chain of one or more amino acid of a fourth type with a fourth detectable moiety, wherein said fourth detectable moiety selectively labels the side chain characterizing said one or more amino acid of a fourth type; in a further embodiment, and wherein before step (c) for each peptide of the plurality, labeling the amino acid side chain of one or more amino acid of a fifth type with a fifth detectable moiety, wherein said fifth detectable moiety selectively labels the side chain characterizing said one or more amino acid of a fifth type; and in a further embodiment, and wherein before step (c) for each peptide of the plurality, labeling the amino acid side chain of one or more amino acid of a sixth type with a sixth detectable moiety, wherein said sixth detectable moiety selectively labels the side chain characterizing said one or more amino acid of a sixth type.

The present disclosure further provides said immediately preceding method for sequencing a plurality of peptides, in one embodiment, wherein the side chain characterizing said one or more amino acid of a first type is positively charged; in a further embodiment, wherein said one or more amino acid of a first type is lysine; in a further embodiment, wherein the side chain characterizing said one or more amino acid of a first type is negatively charged; in a further embodiment, wherein the side chain characterizing said one or more amino acid of a first type is aromatic; in a further embodiment, wherein the side chain characterizing said one or more amino acid of a first type is polar; and in a further embodiment, wherein said one or more amino acid of a first type is cysteine.

The present disclosure further provides said immediately preceding method for sequencing a plurality of peptides, in one embodiment, wherein the cleavage of step (e) is Edman degradation; in one embodiment, wherein the cleavage of step (e) is a digestion; and in one embodiment, wherein the digestion is chemical digestion or enzymatic digestion.

The present disclosure further provides said immediately preceding method for sequencing a plurality of peptides, in one embodiment, wherein the attaching each of said plurality of peptides to a surface of step (c) is attachment of the C-terminus or a side chain of each peptide to the surface; in one embodiment, wherein each of said detectable moieties is selected from the group consisting of a fluorophore, a dye, a quantum dot, a radiolabel, an enzyme and an enzyme substrate; in one embodiment, wherein each of said detectable moieties is a fluorophore; and in one embodiment, wherein after step (i) and before step (j), comparing at least one change or at least one absence of a change in the image identified in step (h) or (i) to a database of fluorescence emission signatures of known protein sequences, further wherein at least one fluorescence emission signature, or part thereof, is the same as the at least one change or the at least one absence of a change in the image of step (j) used for determining the sequence of said peptide.

The present disclosure provides, in one embodiment, a method for sequencing a plurality of peptides in a biological sample comprising: (a) obtaining a biological sample comprising proteins and digesting said biological sample to produce a plurality of peptides; (b) for each peptide of the plurality, labeling the amino acid side chain of one or more amino acid of a first type with a first detectable moiety, wherein said first detectable moiety selectively labels the side chain characterizing said one or more amino acid of a first type; (c) for each peptide of the plurality, labeling the amino acid side chain of one or more amino acid of a second type with a second detectable moiety, wherein said second detectable moiety selectively labels the side chain characterizing said one or more amino acid of a second type; (d) attaching each of said plurality of peptides to a surface such that each peptide is spatially separated enough to allow single-molecule detection; (e) imaging each of said plurality of peptides using single-molecule detection; (f) cleaving each of said plurality of peptides; (g) imaging each of said plurality of peptides using single-molecule detection after the cleavage of step (f); (h) repeating steps (f) to (g) as necessary; (i) comparing the image of step (e) for each of said plurality of peptides with the corresponding image of step (g) and identifying a change or an absence of a change in the image between step (e) and step (g); (j) if further cleavage is performed as in step (h), comparing the image before and corresponding image after each subsequent cleavage step (f) for each of said plurality of peptides and identifying a change or an absence of a change in the image; and (k) determining the sequence of each of said plurality of peptides based on at least one change or at least one absence of a change in the image corresponding to the peptide as identified in step (i) or (j).

The present disclosure provides, in one embodiment, a method for diagnosing a disease or medical condition by sequencing a plurality of peptides in a biological sample comprising: (a) obtaining a biological sample comprising proteins and digesting said biological sample to produce a plurality of peptides; (b) for each peptide of the plurality, labeling the amino acid side chain of one or more amino acid of a first type with a first detectable moiety, wherein said first detectable moiety selectively labels the side chain characterizing said one or more amino acid of a first type; (c) for each peptide of the plurality, labeling the amino acid side chain of one or more amino acid of a second type with a second detectable moiety, wherein said second detectable moiety selectively labels the side chain characterizing said one or more amino acid of a second type; (d) attaching each of said plurality of peptides to a surface such that each peptide is spatially separated enough to allow single-molecule detection; (e) imaging each of said plurality of peptides using single-molecule detection; (f) cleaving each of said plurality of peptides; (g) imaging each of said plurality of peptides using single-molecule detection after the cleavage of step (f); (h) repeating steps (f) to (g) as necessary; (i) comparing the image of step (e) for each of said plurality of peptides with the corresponding image of step (g) and identifying a change or an absence of a change in the image between step (e) and step (g); (j) if further cleavage is performed as in step (h), comparing the image before and corresponding image after each subsequent cleavage step (f) for each of said plurality of peptides and identifying a change or an absence of a change in the image; (k) determining the sequence of each of said plurality of peptides based on at least one change or at least one absence of a change in the image corresponding to the peptide as identified in step (i) or (j); and (l) diagnosing a disease or medical condition based, at least in part, on the sequences determined in step (k) for each of said plurality of peptides.

The present disclosure provides, in one embodiment, a peptide comprising a plurality of differentially labeled amino acid residues, wherein said peptide is attached to a surface; in one embodiment, wherein each of said differentially labeled amino acid residues comprise a differentially labeled side chain; and, in one embodiment, wherein each of said differentially labeled side chains comprise a fluorescent label.

The present disclosure provides, in one embodiment, a method for identifying a peptide comprising: (a) labeling the amino acid side chain of one or more amino acid of a first type with a first detectable moiety, wherein said first detectable moiety selectively labels the side chain characterizing said one or more amino acid of a first type; (b) labeling the amino acid side chain of one or more amino acid of a second type with a second detectable moiety, wherein said second detectable moiety selectively labels the side chain characterizing said one or more amino acid of a second type; (c) attaching said peptide to a surface; (d) imaging said peptide; (e) cleaving said peptide by chemical or enzymatic digestion; (f) imaging said peptide after the cleavage of step (e); (g) repeating steps (e) to (f) as necessary; (h) comparing the image of step (d) with the image of step (f) and identifying any change in the image between step (d) and step (f); (i) if further cleavage is performed as in step (g), comparing the image before and after each subsequent cleavage step (e) and identifying any change in the image; (j) comparing at least one change in the image identified in step (h) or (i) to a database of changes in the images of known protein sequences due to equivalent cleavage; and (k) identifying the peptide.

The present disclosure provides, in one embodiment, a method for sequencing a peptide and determining the presence or absence of a post-translational modification of said peptide comprising: (a) labeling the amino acid side chain of one or more amino acid of a first type with a first detectable moiety, wherein said first detectable moiety selectively labels the side chain characterizing said one or more amino acid of a first type; (b) labeling the amino acid side chain of one or more amino acid of a second type with a second detectable moiety, wherein said second detectable moiety selectively labels the side chain characterizing said one or more amino acid of a second type; (c) labeling said peptide such that a post-translational modification, if present, is labeled in a manner distinct from the labeling of any amino acid side chain; (d) attaching said peptide to a surface; (e) imaging said peptide; (f) cleaving said peptide; (g) imaging said peptide after the cleavage of step (f); (h) repeating steps (f) to (g) as necessary; (i) comparing the image of step (e) with the image of step (g) and identifying a change or an absence of a change in the image between step (e) and step (g); (j) if further cleavage is performed as in step (h), comparing the image before and after each subsequent cleavage step (f) and identifying a change or an absence of a change in the image; (k) comparing at least one change or at least one absence of a change in the image identified in step (i) or (j) to a database of image information of known protein sequences; (l) determining the sequence of the peptide based on the comparison of step (k); and (m) determining the presence or absence of a post-translational modification of said peptide based on the imaging of step (e) of the labeling of a post-translation modification, if present, of step (c).

The present disclosure further provides said immediately preceding method for sequencing a peptide and determining the presence or absence of a post-translational modification of said peptide, in one embodiment, wherein a post-translational modification is a glycosylation and at least one sugar attached to the peptide is oxidized and reacted with a hydrazide fluorophore; and, in one embodiment, wherein a post-translational modification is a phosphorylation and at least one phosphate group attached to the peptide is reacted with 1-ethyl-3-(3-dimethylaminopropyl)-carbodiimide, imidazole and an amine containing fluorophore.

The present disclosure provides, in one embodiment, a method for identifying a plurality of peptides in a biological sample comprising: (a) obtaining a biological sample comprising proteins and digesting said biological sample to produce a plurality of peptides; (b) for each peptide of the plurality, labeling the amino acid side chain of one or more amino acid of a first type with a first detectable moiety, wherein said first detectable moiety selectively labels the side chain characterizing said one or more amino acid of a first type; (c) for each peptide of the plurality, labeling the amino acid side chain of one or more amino acid of a second type with a second detectable moiety, wherein said second detectable moiety selectively labels the side chain characterizing said one or more amino acid of a second type; (d) attaching each of said plurality of peptides to a surface such that each peptide is spatially separated enough to allow single-molecule detection; (e) imaging each of said plurality of peptides using single-molecule detection; (f) cleaving each of said plurality of peptides; (g) imaging each of said plurality of peptides using single-molecule detection after the cleavage of step (f); (h) repeating steps (f) to (g) as necessary; (i) comparing the image of step (e) for each of said plurality of peptides with the corresponding image of step (g) and identifying a change or an absence of a change in the image between step (e) and step (g); (j) if further cleavage is performed as in step (h), comparing the image before and corresponding image after each subsequent cleavage step (f) for each of said plurality of peptides and identifying a change or an absence of a change in the image; and (k) identifying each of said plurality of peptides based on at least one change or at least one absence of a change in the image corresponding to the peptide as identified in step (i) or (j).

In one embodiment, a single-molecule detection of fluorophore-labeled synthetic peptides is disclosed using multiple rounds of standard Edman degradation. Different fluorophores attached to specific amino acid side chains result in the derivation of a peptide's encoded amino acid sequence following image alignments of multiple Edman cycles. Further, the method uses peptide derivatization and immobilization strategies to enable the sequencing and identification of peptides derived from a protein mixture.

In one embodiment, the present invention contemplates a method comprising: a) providing; i) a peptide comprising a plurality of differentially labeled amino acid residues; ii) a mixture comprising components capable of performing Edman degradation; iii) a counting device capable of distinguishing between the differentially labeled amino acid residues; b) counting the differentially labeled amino acid residues on the peptide wherein a first number is generated; c) contacting the peptide with the mixture, wherein a terminal amino acid residue is released from the peptide thereby creating a residual peptide; d) counting the differentially labeled amino acid residues on the residual peptide wherein a second number is generated; e) comparing the first number with the second number, wherein the released terminal amino acid residue is identified. In one embodiment, the method further comprises providing a solid substrate. In one embodiment, the peptide is immobilized to the solid substrate. In one embodiment, the solid substrate comprises a microarray. In one embodiment, the microarray comprises between approximately 10,000-1,000,000 of the immobilized peptides. In one embodiment, the solid substrate comprises a material selected from the group consisting of glass, silicon, and/or quartz. In one embodiment, the counting device comprises an imaging device. In one embodiment, the released terminal amino acid residue comprises an N-terminal amino acid residue. In one embodiment, each of the differentially labeled amino acid residues comprise a differentially labeled side chain. In one embodiment, the differentially labeled side chain comprises a fluorescent label. In one embodiment, the differentially labeled side chain is selected from the group consisting of a hydrophobic side chain, an aromatic side chain, an acidic side chain and a basic side chain. In one embodiment, the method further comprises repeating steps (b)-(e) such that an encoded amino acid sequence is identified. In one embodiment, the method further comprises comparing the encoded amino acid sequence to a proteomic database, wherein a complete amino acid sequence of said peptide is identified. In one embodiment, the hydrophobic side chain comprises a first label. In one embodiment, the aromatic side chain comprises a second label. In one embodiment, the acidic side chain comprises a third label. In one embodiment, the basic side chain comprises a fourth label. In one embodiment, the peptide ranges in length between approximately 10-100 amino acid residues. In one embodiment, the peptide ranges in length between approximately 15-75 amino acid residues. In one embodiment, the peptide ranges in length between approximately 20-50 amino acid residues. In one embodiment, the peptide ranges in length between approximately 25-35 amino acid residues. In one embodiment, the peptide is 30 amino acid residues in length. In one embodiment, the fluorescent label comprises an N-hydroxysuccinimide ester fluorophore. In one embodiment, the fluorescent label comprises a maleimide fluorophore. In one embodiment, the fluorescent label comprises an amine-containing fluorophore. In one embodiment, the fluorescent label comprises a tyrosine-selective reagent. In one embodiment, the fluorescent label comprises a reagent selective for acidic residues (glutamate and aspartate). In one embodiment, the fluorescent label comprises a tryptophan-selective reagent. In one embodiment, the N-hydroxysuccinimide ester fluorophore labels a lysine amino acid residue. In one embodiment, the maleimide fluorophore labels a cysteine side chain. In one embodiment, the amine-containing fluorophore labels a glutamate side chain. In one embodiment, the amine-containing fluorophore labels an aspartate side chain.

In some embodiments of the present invention, the released cyclized terminal amino acid is discarded and the amino acid is identified by image analysis of the post-Edman degradation residual truncated peptide.

In one embodiment, the present invention contemplates a peptide comprising a plurality of differentially labeled amino acid residues. In one embodiment, each of the differentially labeled amino acid residues comprises a differentially labeled side chain. In one embodiment, the differentially labeled side chain comprises a fluorescent label. In one embodiment, the differentially labeled side chain is selected from the group consisting of a hydrophobic side chain, an aromatic side chain, an acidic side chain and a basic side chain. In one embodiment, the hydrophobic side chain comprises a first label. In one embodiment, the aromatic side chain comprises a second label. In one embodiment, the acidic side chain comprises a third label. In one embodiment, the basic side chain comprises a fourth label. In one embodiment, the peptide ranges in length between approximately 10-100 amino acid residues. In one embodiment, the peptide ranges in length between approximately 15-75 amino acid residues. In one embodiment, the peptide ranges in length between approximately 20-50 amino acid residues. In one embodiment, the peptide ranges in length between approximately 25-35 amino acid residues. In one embodiment, the peptide is 30 amino acid residues in length. In one embodiment, the fluorescent label comprises an N-hydroxysuccinimide ester fluorophores. In one embodiment, the fluorescent label comprises a maleimide fluorophores. In one embodiment, the fluorescent label comprises an amine-containing fluorophore. In one embodiment, the fluorescent label comprises a tyrosine-selective reagent. In one embodiment, the fluorescent label comprises a tryptophan-selective reagent. In one embodiment, the N-hydroxysuccinimide ester fluorophore labels a lysine amino acid residue. In one embodiment, the maleimide fluorophore labels a cysteine side chain. In one embodiment, the amine-containing fluorophore labels a glutamate side chain. In one embodiment, the amine-containing fluorophore labels an aspartate side chain.

In one embodiment, the present invention contemplates a kit comprising; a) a first container comprising a first fluorescent label; b) a second container comprising a second fluorescent label; c) a third container comprising a third fluorescent label; d) a fourth container comprising a fourth fluorescent label; e) a fifth container comprising components capable of performing Edman degradation; f) a sixth container comprising components capable of derivatizing peptides for immobilization; g) instructions for attaching the first, second, third and fourth fluorophores to specific amino acid residues on a peptide; and h) instructions for using a counting device to distinguish the first, second, third and fourth fluorescent labels on the peptide. In one embodiment, the fluorescent label comprises a fluorophore. In one embodiment, the fluorophore comprises an N-hydroxysuccinimide ester fluorophores. In one embodiment, the fluorophore comprises a maleimide fluorophores. In one embodiment, the fluorophore comprises an amine-containing fluorophore. In one embodiment, the fluorophore comprises a tyrosine-selective reagent. In one embodiment, the fluorophore comprises a tryptophan-selective reagent. In one embodiment, the N-hydroxysuccinimide ester fluorophore attaches to a lysine amino acid residue. In one embodiment, the maleimide fluorophore attaches to a cysteine side chain. In one embodiment, the amine-containing fluorophore attaches to a glutamate side chain. In one embodiment, the amine-containing fluorophore attaches to an aspartate side chain.

In one embodiment, the present invention contemplates a process for determining at least a portion of amino acid sequence of a plurality of polypeptides in a sample, the process comprising the steps of: (i) digesting said polypeptides into smaller polypeptide sequences; (ii) derivatizing reactive amino acid side chains of said polypeptides with chemoselective reactive fluorophores; (iii) bonding at least some of the plurality of polypeptides of the sample, each at a specific location on a surface; (iv) obtaining an image of said sample; (v) performing a single cycle of Edman degradation during which the N-terminal amino acid moiety from the polypeptides are removed; (vi) repeating steps (iv) through (v) in order to determine at least a portion of the amino acid sequence of the at least some of the polypeptides at the specific locations on the surface via analysis of the image by comparison of the image sequence with probable matches in sequence. In one embodiment, steps (iv) through (v) are repeated. In one embodiment, the digestion is accomplished with proteolytic enzymes. In one embodiment, the proteolytic enzymes comprise: trypsin, chymotrypsin, chymotrypsin B, pancreatopeptidase, carboxypeptidase A, carboxypeptidase B, Endo Glu-C, proteinase K, and mixtures thereof. In one embodiment, the digestion is accomplished with a chemical reaction. In one embodiment, the derivatization of reactive amino acid side chains of said polypeptides with chemoselective reactive fluorophores comprises: lysine side chains reacted with N-hydroxysuccinimide ester fluorophores, cysteine side chains reacted with maleimide fluorophores, tyrosine- and tryptophan-selective reagents, and glutamate and aspartate reacted with N-(3-(Dimethylamino)propyl)-N′-ethylcarbodiimide followed by amine-containing fluorophores. In one embodiment, the fluorophores are mutually exclusive for each different type of amino acid side chain labeled. In one embodiment, step (iv) comprises fluorescence microscopy. In one embodiment, step (iv) comprises total internal reflectance microscopy. In one embodiment, step (iv) comprises photobleaching. In one embodiment, the polypeptide is a protein. In one embodiment, the method further comprises the step of identifying the polypeptide of the sample bound at a specific location on the surface by correlating at least a portion of the amino acid sequence at the specific location with known sequences by performing database searching. In one embodiment, the sequence corresponds to the identified specific fluorophore tagged amino acid side chains. In one embodiment, the method further comprises the step of chemically altering post-translational modifications. In one embodiment, the method further comprises the step of determining the proportion of the amount of polypeptide on the surface to the total amount of polypeptide present in the sample. In one embodiment, the method further comprises the step of determining the amount of the polypeptide on the surface. In one embodiment, the polypeptide is bound to the surface by coupling of native side chains to said surface. In one embodiment, the C-terminus of the polypeptide is bound to the surface.

### EXAMPLES

### Example I

### Simultaneously Detecting Amino Acid Positions in a Plurality of Peptides

This example presents the simultaneous detection of the amino acid positions of 1,000 peptides using SMD following exposure to 30 cycles of Edman sequencing chemistry. Further demonstrated is an ability to identify and distinguish between single peptide molecules that contain between 1 and 5 fluorophores. Our expectation is that this is achievable using standard intensity-based algorithms for determining fluorophore numbers.

### Example II

### Simultaneously Tracking Amino Acid Positions in a Plurality of Peptides

This example presents the simultaneous tracking of the amino acid positions of 1,000 peptides using SMD through 30 cycles of Edman sequencing chemistry. Further demonstrated is the alignment of images from each cycle to identify the loss of fluorophores from these 1,000 peptides at specific cycles. Our expectation is that this is achievable using cross-correlation approaches to minimize X-Y distances between single molecule spots throughout cleavage cycles.

### Example III

### Derivatizing and Immobilizing a Plurality of Peptides

This example presents a method that enables a robust fluorophore-derivatization and immobilization of 1,000 peptides derived from a simple peptide mixture. Further demonstrated is the completion of 30 cycles of Edman sequencing and SMD detection on these 1,000 peptides and derivation of their encoded sequences. Our expectation is that this is achievable using standard attachment chemistries (e.g. NHS, maleimide) and immobilzation reagents.

### Example IV

### Whole Proteome Sequencing with Massively-Parallel Edman Peptide Sequencing

This example presents one embodiment of a peptide sequencing method comprising: 1) a sample preparation phase, 2) a sequencing phase and 3) an analysis phase. In the sample preparation phase, protein and peptide mixtures are digested and derivatized with reactive fluorophores (for visualization) and immobilization reagents. During the sequencing phase, multiple rounds of Edman chemistry and single molecule detection are performed to identify positions that contain labeled amino acids. In the analysis phase, images from the single molecule detection cycles are analyzed to reconstruct an “encoded” sequence for each single molecule. These sequences are used to identify the likely matching peptide sequence from a sequence database.

### 1. Sample Preparation

Proteins from a starting mixture are digested and derivatized with reactive fluorophores to prepare them for sequencing. In addition, immobilization chemistries are added to peptides to facilitate their capture on a substrate for imaging.

a. Digestion

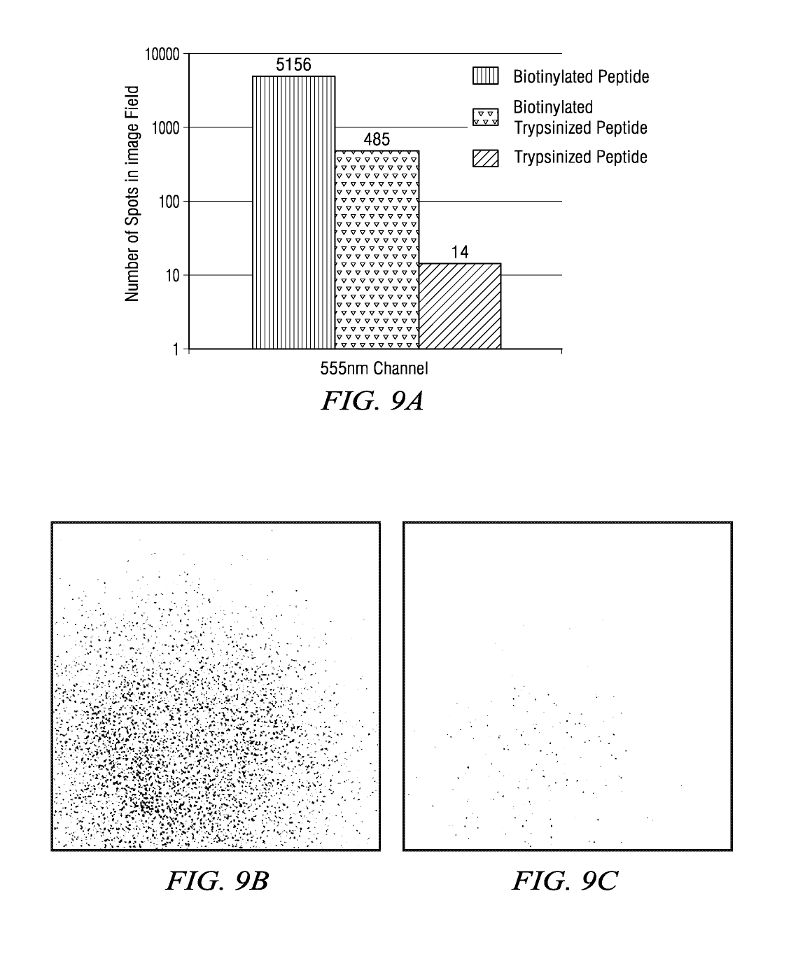

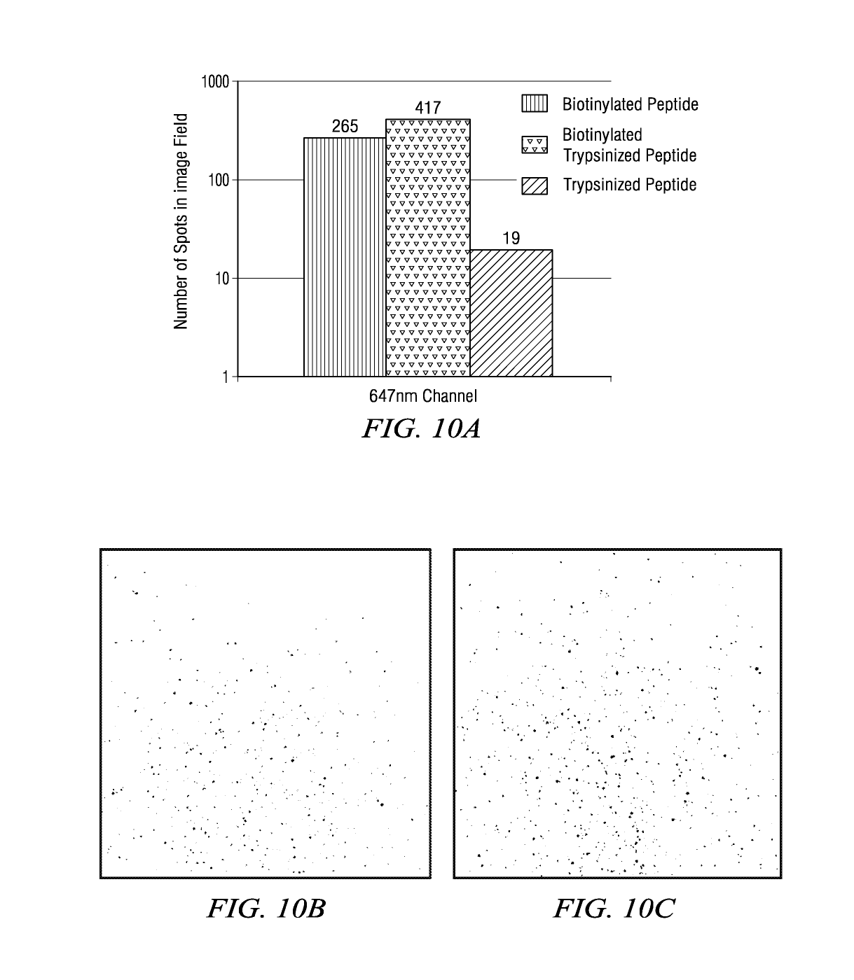

Peptides can be generated from starting proteins by a number of methods. Traditional proteolytic enzymes such as trypsin, chymotrypsin and Endo Glu-C can be used to cleave proteins at specific residues, whereas other proteases (e.g. proteinase K) can be used to generate a pseudo-random mix of peptides. For example, in FIGS. 9-11, 1 nmol of peptide was digested using 200 ng trypsin (60% Acetonitrile, 50 mM Tris-HCl, 20 mM CaCl₂), (PROMEGA). The reaction was incubated for 1 hour at 37° C. and solvents were removed by evaporation for subsequent steps. Alternatively, chemical means can be used to cleave proteins site-specifically. For example, cyanogen bromide could be used to cleave C-terminal of methionine residue, or 2-nitro-5-thiocyanobenzoic acid (NTCB) could be used to cleave N-terminal of cysteine residues.

b. Fluorophore Derivatization

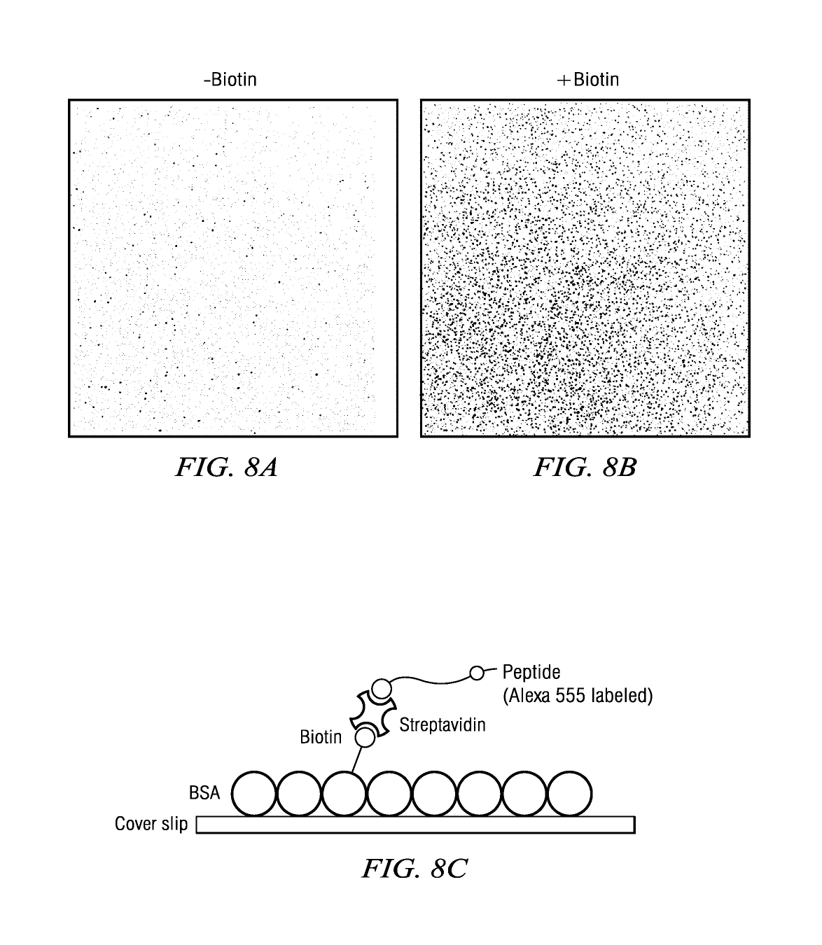

Reactive amino acid side chains are derivatized with chemoselective probes. For example, lysine side chains are reacted with NHS-ester fluorophores, and cysteine side chains are reactive with maleimide fluorophores. For example, in FIGS. 9-11, cysteine side chains were reacted with maleimide ALEXA 647. Similarly, the azide-lysine moieties were coupled to alkyne-ALEXA555 using Cu(I)-mediated Click chemistry.

Additional reactivities can be exploited, including tyrosine- and tryptophan-selective reagents, and acidic side chains (glutamate and aspartate) can be derivatized by treatment with EDC followed by amine-containing fluorophores. Tyrosine-specific reagents have been developed to label tyrosine residues in peptide fragments. Ban et al., “Tyrosine Bioconjugation through Aqueous Ene-Type Reactions: A Click-Like Reaction for Tyrosine” J. Am. Chem. Soc. 132:1523-5 (2010).

At this step, post-translational modifications can be selectively modified. For example, sugar groups in sites of glycosylation can be oxidized (e.g. using sodium periodate) and reactive with fluorophore hydrazides. Sites of phosphorylation can be reacted with EDC and coupled to amine-containing fluorophores.

### 2. Peptide Sequencing

a. Immobilization

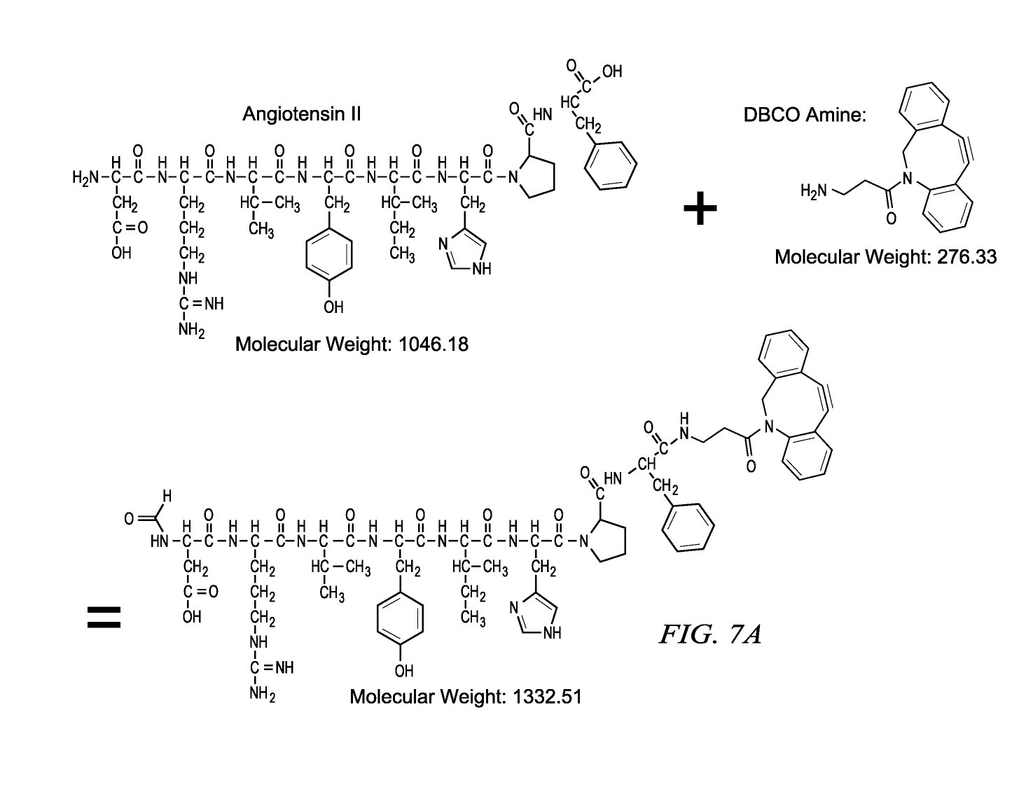

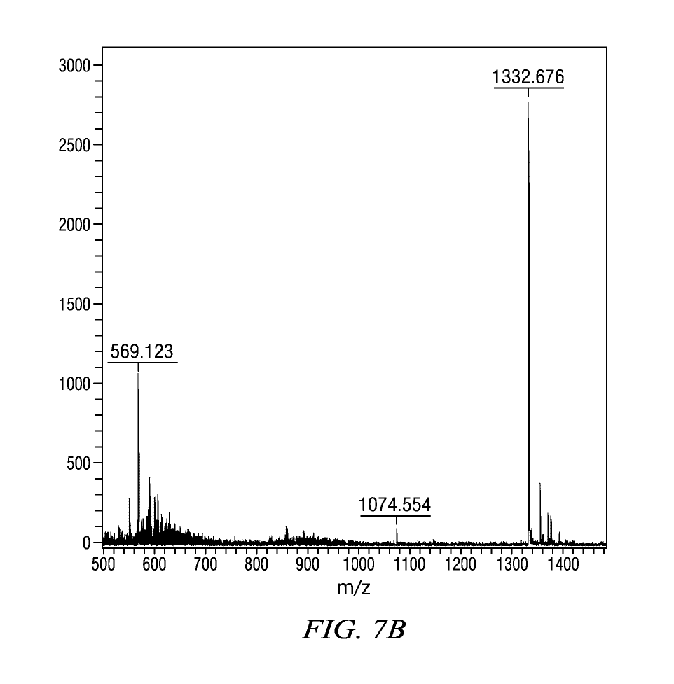

Flow cells are assembled using a aminosilanized coverslip, double-sided adhesive, and a glass slide with drilled inlet and outlet ports. To coat coverslips with aminosilane, they are cleaned thoroughly with two cycles of alternating washes of 100% Ethanol and 1M Potassium Hydroxide. After washing, excess water is removed with an acetone wash. Coverslips are silanized for 2 minutes in a solution of 2% 3-aminopropyltriethoxysilane and acetone by agitation and the reaction is quenched with excess deionized water. Coverslips are dried in a vacuum oven and stored under vacuum until further use. Flow cells are assembled by affixing double side adhesive with a channel cut in the center of the adhesive around the inlet and outlet ports of the glass slide. A silanized coverslip is then affixed to the slide and double side adhesive. Inlet and outlet tubing is glued to the flow cell by inserting the tubing into a rubber O-ring that is glued by epoxy over the inlet or outlet hole of the glass side. The tubing is secured with epoxy as well and cured for 30 minutes. The outlet tube is placed into a 15 ml conical and the inlet tubing is affixed with a luer lock adaptor to be attached to a syringe. Solutions are flowed across the flow cell into the outlet conical tube. To activate the flow cell surface, 2 ml of a 100 mM Sodium Phophate, pH 5.8 buffer is added. While the flow cell is being washed, a 1% BSA solution in 100 mM sodium phosphate is activated with 200 mM EDC (1-ethyl3[3-dimethylamiopropyl]carbodiimide) and 50 mM NHS (N-Hydroxysuccinimide) for 10 minutes at room temperature. Prior to flowing the BSA/EDC/NHS over the flow cell, biotin hydrazide (SIGMA) is added to the mix to a final concentration of 40 μM. 1 ml of the BSA/EDC/NHS/Biotin solution is added to the flow cell and incubated for 1 hour at room temperature. After this step, a layer of biotinylated BSA is covalently attached to the coverslip. Flow cells are washed with 1× Phosphate Buffer Saline (PBS) solution, after which a solution of 15 nM fluorophore conjugated streptavidin (INVITROGEN) is added to the flow cell. The conjugated streptavidin is incubated for 30 minutes at room temperature and washed with 2 ml 1×PBS to remove excess streptavidin. Dye-labeled, biotinylated peptides are then added to flow cells for immobilization. Alternatively, azide amine has been conjugated to the activated BSA (in lieu of biotin), which enables immobilization of peptides with C-terminal alkyne moieties (e.g. FIG. 7A).

Labeled proteins are immobilized on cover slips suitable for TIRF microscopy. Attachment to cover slips is mediated by native side chains (e.g., coupling cysteine-containing peptides to maleimide-containing cover slips) or via immobilization chemistries added in step 1b. For example, click chemistry compatible chemistries can be selectively coupled to tyrosine side chains, enabling the selective immobilization of tyrosine-containing peptides. Finally, peptides containing free C-termini can be modified using oxazalone chemistry to add specific moieties that facilitate derivatization. Kim et al., “C-terminal de novo sequencing of peptides using oxazolone-based derivatization with bromine signature” Anal Biochem. 419(2):211-216 (2011). To modify peptide C-termini for immobilization, the free carboxyl terminus of the peptide must be converted into an activated ester via oxazalone chemistry using a 1:1 mix of acidic anyhydride and formic acid and 100 μmol of an ester-forming leaving group such as (e.g. HOBt or pentafluorophenol). This activation is carried out at 60° C. for 20 min, followed by removal of solvents by evaporation. Following conversion of the C-terminus to an activated ester, the C-terminus is reacted with primary amine-containing compounds under basic conditions of. The amine compounds contain functional groups for non-covalent (biotin and streptavidin) or covalent attachment (click chemistries using an alkyne and azide). Derivatized peptides are purified using C18 ZIP TIPS.

C-terminally derivatized peptides can be subsequently conjugated to specific residues with two or more different fluorophores and immobilized to the streptavidin-activated flow cell. Excess peptide is removed by washing the flow cell with 5 ml of 1×PBS. The flow cell and peptides are ready for imaging or chemistry or degradation by proteolytic or chemical cleavage.

b. Degradation

Edman chemistry cycling of the flow cell is performed to sequentially remove N-terminal residues. Edman chemistry consists of a PITC derivatization step, followed by a cleavage step. Derivatization is performed in the presence of 0.1 M PITC in a 10:5:2:3 mixture of acetonitrile:pyridine:triethyamine:water at 50° C. for 20 minutes. Derivatization reagents are washed away, and cleavage is performed in 1:1 mixture of TEAA:acetonitrile at 75° C. for 10 minutes. Temperature incubations are achieved through direct heating of the cleavage solution, or overtone heating of the sample chamber using laser light. Zhao et al., “Laser-assisted single-molecule refolding (LASR)” Biophys J. 99(6):1925-1931 (2010).

Peptides immobilized on the flow cell can be cleaved with specific reagents added to the flow cell. For example, addition of trypsin enables site-specific cleavage of immobilized peptide at lysine and arginine residues. Alternatively, cyanogen bromide or NTCB can be added to the flow cell to cleave at methionine and cysteine resides, respectively.

c. Imaging

Image analysis is performed to identify cycles in which a fluorophore is lost from a single molecule, indicating a cleavage event and assigning that position of the peptide with the labeled residue. Intensity measurements and/or photobleaching techniques measure the number of fluorophores present in each single molecule throughout cycles of sequencing. Gordon et al., “Single-molecule high-resolution imaging with photobleaching” Proc Natl Acad Sci USA. 101(17):6462-6465 (2004); and Baddeley et al., “Light-induced dark states of organic fluorochromes enable 30 nm resolution imaging in standard media” Biophys J. 96(2):L22-24 (2009). Fluorophores can be individually identified via basic intensity thresholding strategies. A static threshold intensity is established from one example image at which all or most pixels corresponding to fluorophores are above the intensity threshold. All pixels falling below this value are dropped to intensity zero and then all regions of contiguous non-zero pixel values are identified as a fluorophore labeled peptide. Once a standard has been established, this same threshold can be applied to all other images from a given flow cell, thus allowing automated identification of single-molecule events across the entire flow cell. More sophisticated strategies can also be used in which intensity thresholds unique to each image can be established such that only fluorescent regions that fall within the expected intensity range or within a certain number of standard deviations from the mean intensity are counted. This reduces error from issues such as background intensity variation due to molecule density differences and allows a mechanism to discard over-clustered regions that can appear to be a high intensity single molecule event.

Strategies have been developed to count the number of fluorophores on a multiply labeled single molecule. One approach is to integrate the fluorescence intensities from a collection of single molecules in an optical field, fit a Gaussian to the distribution of intensities, and then calculate the probability of a single molecule containing a quantized number of fluorophores using its observed intensity and the Gaussian fit. Mutch et al., “Deconvolving single-molecule intensity distributions for quantitative microscopy measurements” Biophys J. 92(8):2926-2943 (2007). Alternatively, fluorophores can be counted by sequentially photobleaching a field by incrementally increasing excitation intensity and observing how many fluorophores remain in a collection of single molecules following each photobleaching step. Ulbrich et al., “Subunit counting in membrane-bound proteins” Nat Methods 4(4):319-321 (2007).

Prior to analysis, images of the same region across different channels are aligned to compensate for small X/Y or rotational translations. Aligning fluorophore spots between different channels is critical so that the different labeled residues may be attributed to the same peptide. This image registration is accomplished by image cross-correlation in the Fourier domain and iteratively performing translations of the physical space of one of the images until an acceptable degree of alignment is achieved. Currently, this is accomplished using open-source Insight Segmentation and Registration Toolkit (ITK) libraries (http://www.itk.org/).

Once the images across channels are aligned, the locations of single peptides recorded, and the number of fluorophores per molecule identified, encoded sequences can be generated. Using either Edman degradation or sequential cleavage, one or more residues may be removed from the end of the peptide. After each step, images for each channel are collected, aligned, and analyzed for fluorescently labeled peptides as previously described. The images throughout the degradation/cleavage process allow individual peptide intensity to be compared at each step. A probability can be assigned to the event of a loss of one or more particular labeled residues from a peptide based on the channel and the observed decrease in intensity. Little or no intensity change indicates loss of only unlabeled residues. For each peptide identified on the image field, this information is compiled throughout the entire degradation process. For example, assume a peptide of sequence A-C-Y-C (SEQ ID No: 6) with cysteine residues labeled with a ALEXA555 and tyrosine residues labeled with ALEXA647 undergoing Edman degradation. Loss of the first alanine should result in no intensity drop in any channel of the imaged peptide, and so it is noted that an unlabeled residue (i.e. not C or Y) is at that position. Next, the loss of one of the cysteine residues should be accompanied by a roughly 50% drop of intensity of the peptide in the 555 nm channel indicating that a cysteine was removed. Continuing degradation, loss of all signal from that particular peptide in the 647 nm channel followed by loss of all signal from that same peptide in the 555 nm channel informs that the last two residues are a tyrosine followed by another cysteine.

To collect data for large numbers of molecules, multiple fields from a set of immobilized molecules can be captured by raster-scanning the microscope stage and collecting images for each position.

### 3. Analysis

Encoded sequences derived from image analysis are used in an alignment step to identify probable peptide sequence matches. This step is analogous to peptide or DNA sequence matching to a sequence database, except the encoded sequences contain extensive missing information. For example, a typical 30 position sequence will contain 5-10 sites where the residue is known unambiguously, and other positions will be “placeholder” positions, i.e. the identity of the residue at this position is not known definitively, but cannot be one of the residues that was initially modified. In this way, the identities of the known residues as well as their relative positions are informative and can be used during sequence alignment.

Encoded sequences can be used in existing dynamic programming sequence alignment algorithms (e.g. Smith-Waterman) to identify probable matches in a protein sequence database. These algorithms will treat “placeholder” positions as neutral with regard to scoring, such that typical scores from an alignment traceback will be lower than similar traditional sequence alignment approaches. Statistical approaches can permit a robust alignment in the face of false-positive “insertions” and “deletions” created by inefficient derivatization or Edman cleavage.

Alternatively, the “optical transitions” generate by sequential cleavage analysis can be matched to databases of known proteins and peptides. This approach essentially measures the amino acid composition of immobilized peptides (e.g. 4 cysteines, 2 lysines and 6 tyrosines) and searches for peptides in a database that have the same or similar composition. Sequential analysis further narrows the search space by eliminating matches that do not undergo subsequent cleavage steps.

### Example V

### Sequencing by Edman Degradation

A model system has been designed to follow the nature of protein or protein fragments that undergo Edman degradation (i.e. sequential removal N-terminal residues). A small peptide with a labeled lysine residue at the N-Terminus can be used to determine loss of fluorescence over time when exposed to Edman degradation conditions.

NH2-K(AF647)-GSGCSGSG-K(biotin)-amide (SEQ ID No: 7)

AF647=ALEXA FLUOR 647

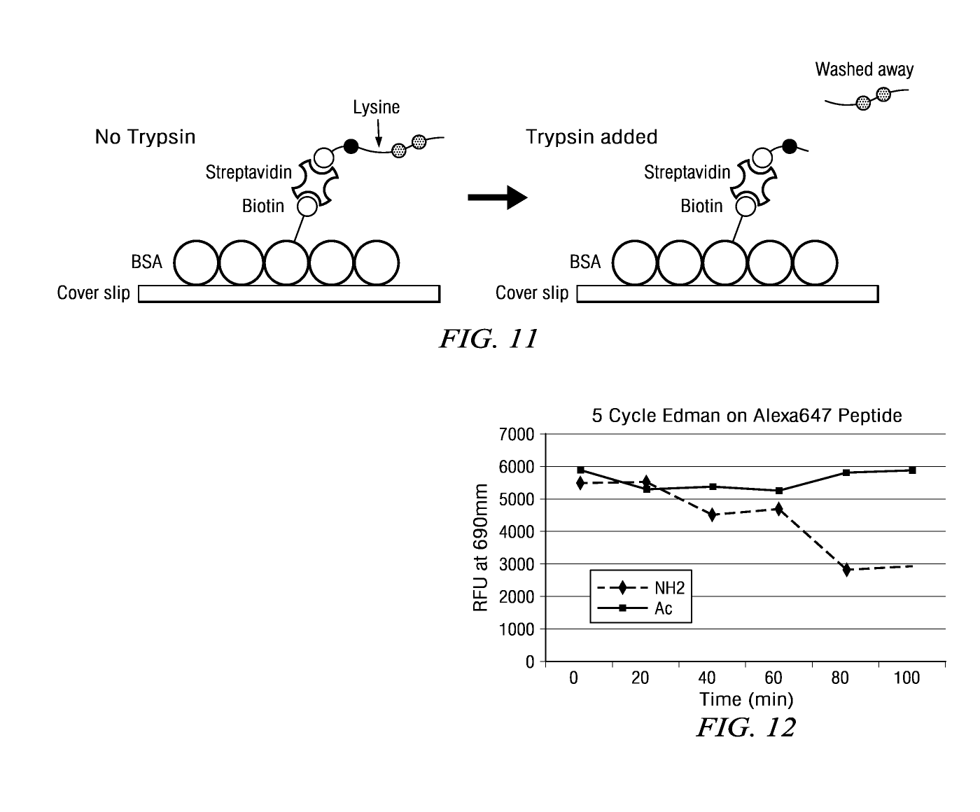

This peptide was exposed to Edman conditions over time, and the loss of fluorescence was observed as a loss of fluorescence on a fluorometer. The C-terminus of the peptide is conjugated with a biotin moiety to use for capture of a small amount of peptide through a time course. At each time point, a small amount of peptide is removed from the Edman reaction and captured using streptavidin magnetic beads. Capturing the peptide allows for removal of free labeled lysines that are in solution. The peptides' fluorescence can be measured on a fluorometer at the ALEXA FLUOR excitation of 647 nm. Over time the peptide captured at various time points loses fluorescence as the Edman reaction goes to completion (FIG. 12).

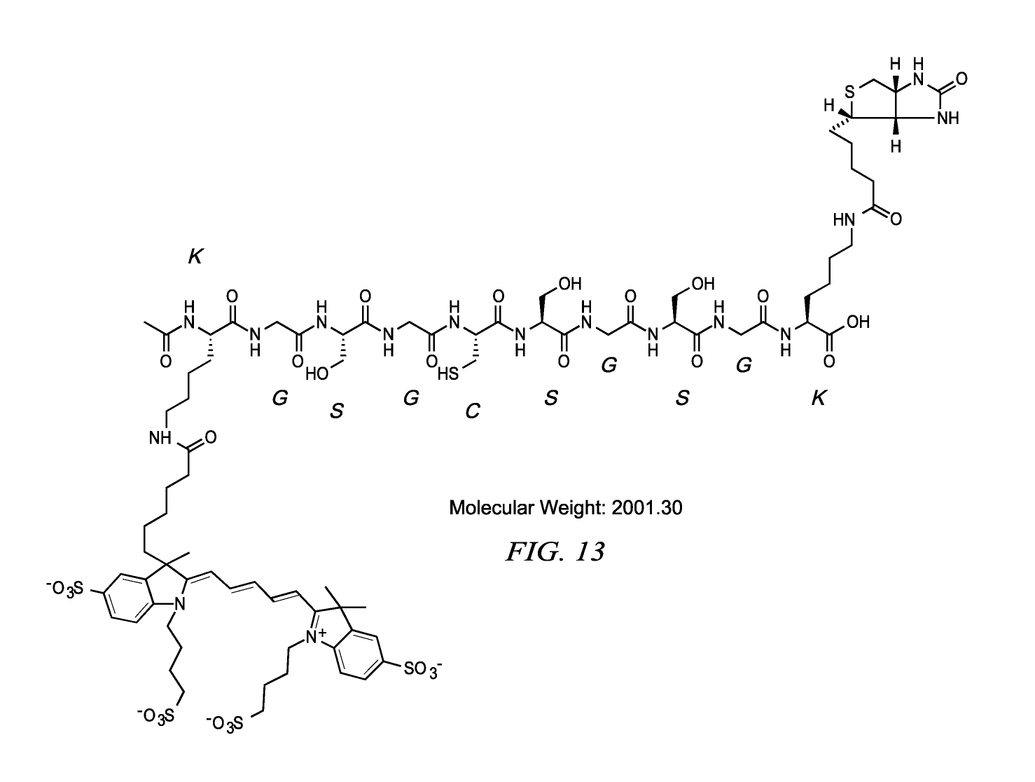

As a control for this model system, a peptide was generated in which the N-terminus was acetylated, blocking the peptide from undergoing Edman chemistry (FIG. 13).

Ac-K(AF647)-GSGCSGSG-K(biotin)-amide SEQ ID No: 8)
Ac = Acetylated
AF647 = ALEXA FLUOR 647

The acetylated peptide was not susceptible to Edman degradation and maintained fluorescence over time (FIG. 13).

This model system can be used to optimize the overall Edman reaction to show degradation of multiple residues for protein or protein fragments.

A detailed protocol follows for the Edman degradation of the model peptideProtocol and Results:
1) Peptides were synthesized by the University of Colorado Denver Peptide and Protein Chemistry Core. Peptides were received in lyophilized form. The powdered peptides were stored protected from light at 4° C.
2) Peptides were resuspended and the concentrations were calculated based on extinction coefficients.
3) 1 nmol of peptide was added to 200 μl of buffer (10% acetonitrile in 1M triethylammonium acetate buffer) in a glass vial. The final concentration of peptide was approximately 5 pmol/μl.
4) 10 μl of phenyisothiacyanate (PITC) was added to the above reaction and mixed thoroughly.
5) The reaction was incubated in a heat block at 70° C. for 10 minutes. The vial was removed and placed on ice for an additional 10 minutes.
6) A 10 μl aliquot with ˜50 pmol PITC-derivatize peptide was removed from the glass vial and placed in a 1.7 ml microcentrifuge tube.
7) The 10 μl aliquot was neutralized with 1 mL of 1× Tris EDTA.
8) To purify the peptide, 50 uL of magnetic streptavidin beads (from LIFE TECHNOLOGIES) were added.
9) After a 15 minute incubation, the beads isolated by magnetic separation of the tube and the supernatant containing released ALEXA 647 lysine residues was removed.
10) Beads were washed and saved for analysis.
11) Steps 6-10 were repeated for the remaining time points. For this experiment, 20 minute time points were collected for a total of 100 minutes.
12) To measure remaining fluorescence for each time point, 2 μl of purified peptide was measured from each time point on a Nanodrop 3000 or equivalent fluorometer at excitation 597 nm and emission of 690 nm.

Both the free N-terminus peptide and the acetylated peptide were exposed to Edman reagent allowing for the N-terminal residue to be removed. The free N-terminus peptide had a lysine residue at the N-terminus labeled with an ALEXA 647 fluorophore. As this peptide was exposed to Edman reagent, Edman degradation removed the N-terminal lysine residue and a loss of fluorescence was observed over time as seen in our results. Alternatively, the peptide with an acetylated N-terminus also contained a N-terminal lysine labeled with ALEXA 647 fluorophore. However Edman degradation could not degrade this peptide's N-terminus because the N-terminus was protected by acetylation. Therefore when exposed to Edman reagent, over time the acetylated peptide had no loss of fluorescence as also seen in our results (FIG. 12).

The results of FIG. 12 indicate that a free N-terminal peptide can be exposed to Edman degradation and will lose the N-terminal residue over time. This can be applied to our sequencing approach by applying the same principle to protein peptide fragments. Protein fragments can be labeled at particular residues with different fluorophores and anchored to a solid surface. The fragments can then be exposed to Edman degradation releasing one N-terminal residue per Edman cycle. As the labeled residues are release at different cycles, the fluorescent pattern of the peptide after each Edman cycle will change indicating what type of residue was lost..

### Example VI

### C-Terminal Derivatization of Peptide

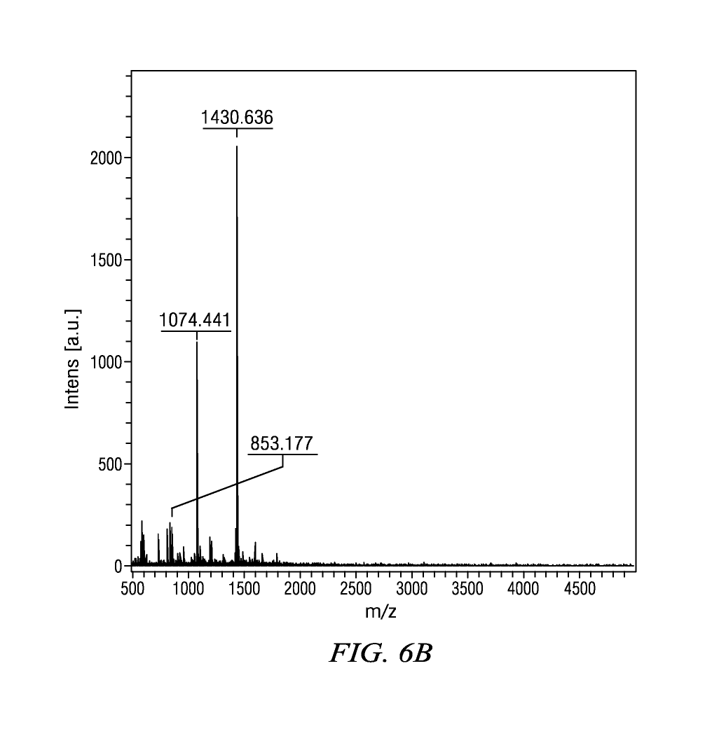

A model peptide (e.g. Angiotensin II) was derivatized at its C-terminus using oxazalone chemistry to add biotin (FIG. 6A) and DBCO (FIG. 7A) moieties. The presently disclosed work has found that using the same peptide, oxazalone-mediated C-terminal attachment chemistry can be used with these moieties to anchor peptides to a solid surface. One system, presently developed, uses a biotin moiety on the C-terminal end of the peptide, which can then be used with streptavidin coated flow cells to anchor the peptide to the flow cell surface. To accomplish this, a biotin amine compound is added to the model peptide, Angiotensin II, using C-terminal chemistry. To determine if the biotin has been added to the C-terminal end of the peptide, MALDI mass spectrometry was used to confirm attachment. Formylated angiotensin II has a molecular weight of 1074, whereas the biotinylated derivative has mass 1430 (FIG. 6B). Similarly, addition of DBCO amine to the peptide was performed using identical oxazalone chemistry and its mass (1332.6 m/z) was confirmed by MALDI mass spectrometry (FIG. 7B).

Once the peptide is reacted with a 1:1 mixture of acetic anhydride and formic acid and 100 μmol of pentafluorphenol, an activated ester is formed at the C-terminus. The ester can then be reacted with an amine biotin compound generating a peptide with a new molecular mass of 1430.6 (FIG. 6A).

Following this step, the C-terminus contains a biotin moiety. The N-terminus will become formylated as well as any primary amines through the C-terminal chemistry. The formylated peptide alone has a mass of 1074.19, indicating it has been activated by the chemistry.

Once the biotin moiety has been added to the peptide, the peptide can be used in downstream processes by adhering the peptide to streptavidin coated flow cell surfaces. Fluorescent labels can be added before or after C-terminal chemistry and then using the biotin on the C-terminal of the peptide, the peptide can be anchored to a flow cell surface to observe fluorescent signals from the peptide.

A detailed protocol for C-terminal activation follows:
1) Angiotensin II peptide (ANASPEC, INC) was resuspended at a concentration of 5 mg/ml in distilled water.
2) 1 nmol peptide was dried in a Speed Vac to completeness.
3) 200 uL of a 1:1 ratio of acetic anhydride and formic acid was added to the peptide, followed by the addition of 100 μmol PfP was added to the v-vial and incubated for 20 minutes at 60° C. with no mixing. The v-vial was cooled at room temperature for 10 minutes.
4) Solvent was removed by Speed Vac.
5) 20 μl of 1:1 ACN:TEA was added followed by 20 μl of 50 μM amine-PEG2-biotin (from PIERCE BIOTECHNOLOGY) or DBCO-amine (SIGMA). The peptide was resuspended thoroughly by pipetting up and down. The biotin/DBCO-amine was in 1000-fold excess of peptide (1 mmol final).
6) This reaction incubated at room temperature for 2 hours with shaking (660 rpms in a thermomixer).
7) Solvent was removed in a Speed Vac.
8) 10 μl of 1% trifluoracetic acid (TFA) was added and the peptide was allowed to resuspend overnight at room temperature.
9) The peptide was purified using C18 ZIP TIPS. The manufacturer's protocol was followed and derivatized peptide was eluted in 7 μl of 60% ACN in 0.1% TFA.
10) Masses of derivatized peptides were determined by MALDI mass spectrometry.

Our model peptide was used in this experiment to determine if moieties such as biotin can be derivatized to peptides. We observed that our starting peptide had a molecular mass of 1046.7 as scanned by MALDI.

We performed the addition of a DBCO moiety to our model peptide. In doing this we saw the expected mass shift from 1046 to 1332 indicating that our C-terminal chemistry has successful added a DBCO species to the C-terminus (FIG. 7B). Note that a molecular weight of 1074 is also observed in the spectra of FIG. 7B, which is the formylated peptide from the oxazalone activation step of our derivatization process. Since no starting peptide molecular mass of 1046 is observed in FIG. 7B, this indicates that our activated ester step goes to completion. The actual activated ester species is unstable and not observable by MALDI.

Finally, we confirmed addition of a biotin species to the C-terminus of our model peptide by observing a molecular mass shift from 1046 to 1430 as seen by MALDI (FIG. 6B).

FIG. 6B shows that derivatization of the model peptide with the biotin species was more than 50% of the starting material. This efficiency can be improved by allowing the biotin amine compound to react with the activated ester for a longer time to allow the reaction to go more to completion. This chemistry can now be used to anchor peptides to solid surfaces for single molecule observations. Fluorescent dyes can be conjugated to peptides before or after C-terminal derivatization. Once the peptides are dye labeled and biotin labeled, they can be applied to a streptavidin coated solid surface and anchored to that surface by a streptavidin-biotin affinity interaction. That interaction can be used to observe single molecule downstream processes.

### Example VII

### Peptide Identification and Sequencing in Medical Diagnostics and Treatment

This example presents the diagnosing of a disease or medical condition by sequencing a plurality of peptides in a biological sample from a patient. A biological sample obtained from a patient is prepared by digestion of proteins to produce a plurality of peptides. Each of the plurality of peptides is differentially labeled as herein disclosed. The plurality of peptides are attached to a surface, by functionalizing the C-terminals of the peptides with biotin and attaching to a streptavidin surface, or other means as disclosed herein. The plurality of peptides is imaged by single molecule detection fluorescence microcopy, as in FIG. 9B, or other means as disclosed herein. The plurality of peptides is cleaved by Edman degradation or sequential cleavage. A second image is taken of the peptides, as in FIG. 9C, and an optical transition or absence of an optical transition is detected. Further, cleavage and imaging is performed as necessary to provide, after bioinformatics analysis, the sequence and/or identity of a sufficient number of peptides, thereby providing useful information on which to base, at least in part, a diagnosis of a disease or condition. Based on this diagnosis, a treatment for the patient is recommended and performed.

1. A method for sequencing one or more than one peptide comprising:
a. labeling the amino acid side chain of one or more amino acid of a first type with a first detectable moiety, wherein said first detectable moiety selectively labels the side chain characterizing said one or more amino acid of a first type;
b. labeling the amino acid side chain of one or more amino acid of a second type with a second detectable moiety, wherein said second detectable moiety selectively labels the side chain characterizing said one or more amino acid of a second type;
c. attaching said peptide to a surface;
d. imaging said peptide;
e. cleaving said peptide;
f. imaging said peptide after the cleavage of step (e);
g. repeating steps (e) to (f) as necessary;
h. comparing the image of step (d) with the image of step (f) and identifying a change or an absence of a change in the image between step (d) and step (f);
i. if further cleavage is performed as in step (g), comparing the image before and after each subsequent cleavage step (e) and identifying a change or an absence of a change in the image; and
j. determining the sequence of the peptide based on at least one change or at least one absence of a change in the image identified in step (h) or (i).

2. The method of claim 1, wherein after step (b) and before step (c) labeling the amino acid side chain of one or more additional type of amino acid with one or more additional detectable moiety, wherein each additional detectable moiety selectively labels the side chain characterizing said one or more additional type of amino acid such that each detectable moiety is selective for only one type of amino acid.

3-5. (canceled)

6. The method of claim 1, wherein the side chain characterizing said one or more amino acid of a first type is positively charged.

7. The method of claim 6, wherein said one or more amino acid of a first type is lysine.

8. The method of claim 1, wherein the side chain characterizing said one or more amino acid of a first type is negatively charged.

9. The method of claim 1, wherein the side chain characterizing said one or more amino acid of a first type is aromatic.

10. The method of claim 1, wherein the side chain characterizing said one or more amino acid of a first type is polar.

11. The method of claim 10, wherein said one or more amino acid of a first type is cysteine.

12. The method of claim 1, wherein the cleavage of step (e) is Edman degradation.

13. The method of claim 1, wherein the cleavage of step (e) is a digestion.

14. The method of claim 13, wherein the digestion is chemical digestion or enzymatic digestion.

15. The method of claim 1, wherein the attaching said peptide to a surface of step (c) is attachment of the C-terminus or a side chain of said peptide to the surface.

16. The method of claim 1, wherein each of said detectable moieties is selected from the group consisting of a fluorophore, a dye, a quantum dot, a radiolabel, an enzyme and an enzyme substrate.

17. The method of claim 16, wherein each of said detectable moieties is a fluorophore.

18. The method of claim 17, wherein after step (i) and before step (j), comparing at least one change or at least one absence of a change in the image identified in step (h) or (i) to a database of fluorescence emission signatures of known protein sequences, further wherein at least one fluorescence emission signature, or part thereof, is the same as the at least one change or the at least one absence of a change in the image of step (j) used for determining the sequence of the peptide.

19-41. (canceled)

42. A method for identifying a peptide comprising:
a. labeling the amino acid side chain of one or more amino acid of a first type with a first detectable moiety, wherein said first detectable moiety selectively labels the side chain characterizing said one or more amino acid of a first type;
b. labeling the amino acid side chain of one or more amino acid of a second type with a second detectable moiety, wherein said second detectable moiety selectively labels the side chain characterizing said one or more amino acid of a second type;
c. attaching said peptide to a surface;
d. imaging said peptide;
e. cleaving said peptide by chemical or enzymatic digestion;
f. imaging said peptide after the cleavage of step (e);
g. repeating steps (e) to (f) as necessary;
h. comparing the image of step (d) with the image of step (f) and identifying any change in the image between step (d) and step (f);
i. if further cleavage is performed as in step (g), comparing the image before and after each subsequent cleavage step (e) and identifying any change in the image;
j. comparing at least one change in the image identified in step (h) or (i) to a database of changes in the images of known protein sequences due to equivalent cleavage; and
k. identifying the peptide.

43. A method for sequencing a peptide and determining the presence or absence of a post-translational modification of said peptide comprising:
a. labeling the amino acid side chain of one or more amino acid of a first type with a first detectable moiety, wherein said first detectable moiety selectively labels the side chain characterizing said one or more amino acid of a first type;
b. labeling the amino acid side chain of one or more amino acid of a second type with a second detectable moiety, wherein said second detectable moiety selectively labels the side chain characterizing said one or more amino acid of a second type;
c. labeling said peptide such that a post-translational modification, if present, is labeled in a manner distinct from the labeling of any amino acid side chain;
d. attaching said peptide to a surface;
e. imaging said peptide;
f. cleaving said peptide;
g. imaging said peptide after the cleavage of step (f);
h. repeating steps (f) to (g) as necessary;
i. comparing the image of step (e) with the image of step (g) and identifying a change or an absence of a change in the image between step (e) and step (g);
j. if further cleavage is performed as in step (h), comparing the image before and after each subsequent cleavage step (f) and identifying a change or an absence of a change in the image;
k. comparing at least one change or at least one absence of a change in the image identified in step (i) or (j) to a database of image information of known protein sequences;
l. determining the sequence of the peptide based on the comparison of step (k); and
m. determining the presence or absence of a post-translational modification of said peptide based on the imaging of step (e) of the labeling of a post-translation modification, if present, of step (c).

44. The method of claim 43, wherein a post-translational modification is a glycosylation and at least one sugar attached to the peptide is oxidized and reacted with a hydrazide fluorophore.

45. The method of claim 43, wherein a post-translational modification is a phosphorylation and at least one phosphate group attached to the peptide is reacted with 1-ethyl-3-(3-dimethylaminopropyl)-carbodiimide, imidazole and an amine containing fluorophore.

46-107. (canceled)

108. The method of claim 1, wherein said method further comprises obtaining a biological sample comprising proteins and digesting said biological sample to produce one or more than one peptide.
